(19) United States
(12) Patent Application Publication
(10) Pub. No.: US 2018/0212180 A1
(43) Pub. Date: Jul. 26, 2018

(54) POLYMERIC CHARGE TRANSFER LAYER AND ORGANIC ELECTRONIC DEVICE CONTAINING SAME

(71) Applicant: Dow Global Technologies LLC, Midland, MI (US)

(72) Inventors: Robert David Grigg, Midland, MI (US); Liam P. Spencer, Manvel, TX (US); John W. Kramer, Midland, MI (US); Chun Liu, Midland, MI (US); Sukrit Mukhopadhyay, Midland, MI (US); David D. Devore, Midland, MI (US); Shaoguang Feng, Shanghai (CN); Jichang Feng, Shanghai (CN); Minrong Zhu, Shanghai (CN)

(21) Appl. No.: 15/744,888

(22) PCT Filed: Aug. 21, 2015
(86) PCT No.: PCT/CN15/87747

§ 371 Date: Jan. 15, 2018

(51) Int. Cl. H01L 51/50; H01L 51/00; C07D 209/82; H01L 51/52

(52) U.S. Cl. CPC H01L 51/5064; H01L 51/0072; H01L 51/006; C07D 209/82; H01L 51/5072; H01L 51/0051; H01L 51/5221; H01L 51/5206; H01L 51/5012

(57) ABSTRACT

The present invention provides a polymeric charge transfer layer composition comprising a polymer comprising, as polymerized units, at least one Monomer A and at least one Monomer B. It further provides an organic light emitting device and an organic electronic device comprising the polymeric charge transfer layer.

### FIELD OF THE INVENTION

The present invention relates to a polymeric charge transfer layer composition comprising a polymer comprising, as polymerized units, Monomer A and Monomer B. The present invention further relates to an organic electronic device, especially, a light emitting device containing the polymeric charge transfer layer.

### INTRODUCTION

Organic electronic devices are devices that carry out electrical operations using at least one organic material. They are endowed with advantages such as flexibility, low power consumption, and relatively low cost over conventional inorganic electronic devices. Organic electronic devices usually include organic light emitting devices, organic solar cells, organic memory devices, organic sensors, organic thin film transistors, and power generation and storage devices such as organic batteries, fuel cells, and organic supercapacitors. Such organic electronic devices are prepared from hole injection or transportation materials, electron injection or transportation materials, or light emitting materials.

A typical organic light emitting device is an organic light emitting diode (OLED) having a multi-layer structure, and typically includes an anode, and a metal cathode. Sandwiched between the anode and the metal cathode are several organic layers such as a hole injection layer (HIL), a hole transfer layer (HTL), an emitting layer (EL), an electron transfer layer (ETL) and an electron injection layer (EIL). New material discovery for ETL and HTL in OLEDs have been targeted to improve device performance and lifetimes. In the case of HTL layer, as a typical polymeric charge transfer layer, the process by which the layer is deposited is critical for its end-use application. Methods for depositing HTL layer, in small display applications, involve evaporation of a small organic compound with a fine metal mask to direct the deposition. In the case of large displays, this approach is not practical from a material usage and high throughput perspective. With these findings in mind, new processes are needed to deposit HTLs that satisfy these challenges, and which can be directly applied to large display applications.

One approach that appears promising is a solution process which involves the deposition of a small molecule HTL material attached with crosslinking or polymerization moiety. Solution process based methods include spin-coating, inkjet printing, and screen printing which are well-known in the art. There have been extensive efforts in this area, along these lines; however, these approaches have their own shortcomings. In particular, the mobility of the charges in the HTL becomes reduced, as a result of crosslinking or polymerization chemistry. This reduced hole mobility leads to poor device lifetime.

Therefore, it is still desired to provide new polymeric charge transfer layer compositions for organic electronic devices, specifically for organic light emitting devices, organic solar cells, or organic memory devices with improved device lifetime.

### SUMMARY OF THE INVENTION

The present invention provides a polymeric charge transfer layer composition comprising a polymer comprising, as polymerized units, at least one Monomer A and at least one Monomer B; wherein Monomer A has Structure A-I:

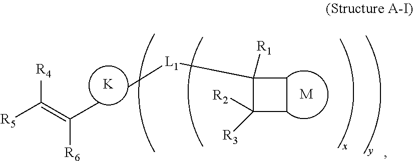

or Structure A-II:

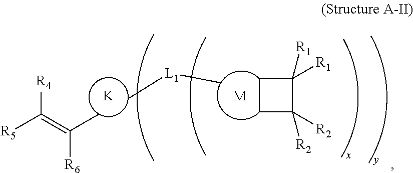

K and M are each independently selected from substituted or unsubstituted aromatic moiety, substituted or unsubstituted heteroaromatic moiety, carbonyl moiety, and carboxy moiety; and

L₁ is selected from heteroatom, aromatic moiety, heteroaromatic moiety, C₁-C₁₀₀ hydrocarbyl, C₁-C₁₀₀ substituted hydrocarbyl, C₁-C₁₀₀ heterohydrocarbyl, and C₁-C₁₀₀ substituted heterohydrocarbyl; and

R₁ through R₆ are each independently selected from hydrogen, deuterium, hydrocarbyl, substituted hydrocarbyl, heterohydrocarbyl, substituted heterohydrocarbyl, halogen, cyano, aryl, substituted aryl, heteroaryl, substituted heteroaryl; and

x and y are each independently an integer from 1 to 10; and

Monomer B has Structure B:

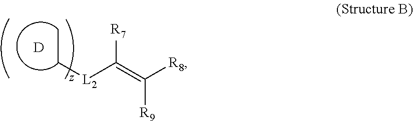

D is selected from aromatic moiety, heteroaromatic moiety, C₁-C₁₀₀ hydrocarbyl, C₁-C₁₀₀ substituted hydrocarbyl, C₁-C₁₀₀ heterohydrocarbyl, and C₁-C₁₀₀ substituted heterohydrocarbyl; and

L₂ may be absent, or is selected from aromatic moiety, heteroaromatic moiety, carboxy moiety, and carbonyl moiety; and

R₇ through R₉ are each independently selected from hydrogen, deuterium, hydrocarbyl, substituted hydrocarbyl, heterohydrocarbyl, substituted heterohydrocarbyl, halogen, cyano, aryl, substituted aryl, heteroaryl, substituted heteroaryl; and

z is an integer from 1 to 10.

The present invention further provides an organic light emitting device and an organic electronic device comprising the polymeric charge transfer layer.

### DETAILED DESCRIPTION OF THE INVENTION

The polymeric charge transfer layer composition of the present invention comprises a polymer and an optional additive. The polymer comprises, as polymerized units, at least one Monomer A, and at least one Monomer B.

The Polymer

The polymer comprises Monomer A having the following Structure A-I:

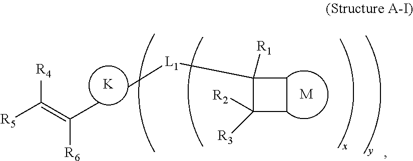

or Structure A-II:

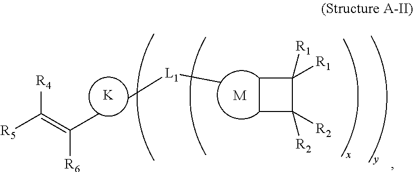

wherein K and M are each independently selected from substituted or unsubstituted aromatic moiety, substituted or unsubstituted heteroaromatic moiety, carbonyl moiety, and carboxy moiety; and

wherein L₁ is selected from heteroatom, aromatic moiety, heteroaromatic moiety, C₁-C₁₀₀ hydrocarbyl, C₁-C₁₀₀ substituted hydrocarbyl, C₁-C₁₀₀ heterohydrocarbyl, and C₁-C₁₀₀ substituted heterohydrocarbyl; and

wherein R₁ through R₆ are each independently selected from hydrogen; deuterium; hydrocarbyl such as C₁-C₁₀₀ hydrocarbyl, preferably C₃-C₁₀₀ hydrocarbyl, more preferably C₁₀-C₁₀₀ hydrocarbyl, even more preferably C₂₀-C₁₀₀ hydrocarbyl, and most preferably C₃₀-C₁₀₀ hydrocarbyl; substituted hydrocarbyl such as C₁-C₁₀₀ substituted hydrocarbyl, preferably C₃-C₁₀₀ substituted hydrocarbyl, more preferably C₁₀-C₁₀₀ substituted hydrocarbyl, even more preferably C₂₀-C₁₀₀ substituted hydrocarbyl, and most preferably C₃₀-C₁₀₀ substituted hydrocarbyl; heterohydrocarbyl such as C₁-C₁₀₀ heterohydrocarbyl, preferably C₃-C₁₀₀ heterohydrocarbyl, more preferably C₁₀-C₁₀₀ heterohydrocarbyl, even more preferably C₂₀-C₁₀₀ heterohydrocarbyl, and most preferably C₃₀-C₁₀₀ heterohydrocarbyl; substituted heterohydrocarbyl such as C₁-C₁₀₀ substituted heterohydrocarbyl, preferably C₃-C₁₀₀ substituted heterohydrocarbyl, more preferably C₁₀-C₁₀₀ substituted heterohydrocarbyl, even more preferably C₂₀-C₁₀₀ substituted heterohydrocarbyl, and most preferably C₃₀-C₁₀₀ substituted heterohydrocarbyl; halogen; cyano; aryl such as C₅-C₁₀₀ aryl, preferably C₆-Coo aryl, more preferably C₁₀-C₁₀₀ aryl, even more preferably C₂₀-C₁₀₀ aryl, and most preferably C₃₀-C₁₀₀ aryl; substituted aryl such as C₅-C₁₀₀ substituted aryl, preferably C₆-C₁₀₀ substituted aryl, more preferably C₁₀-C₁₀₀ substituted aryl, even more preferably C₂₀-C₁₀₀ substituted aryl, and most preferably C₃₀-C₁₀₀ substituted aryl; heteroaryl such as C₅-C₁₀₀ heteroaryl, preferably C₆-C₁₀ heteroaryl, more preferably C₁₀-C₁₀₀ heteroaryl, even more preferably C₂₀-C₁₀₀ heteroaryl, and most preferably C₃₀-C₁₀₀ heteroaryl; substituted heteroaryl such as C₅-C₁₀₀ substituted heteroaryl, preferably C₆-C₁₀₀ substituted heteroaryl, more preferably C₁₀-C₁₀₀ substituted heteroaryl, even more preferably C₂₀-C₁₀₀ substituted heteroaryl, and most preferably C₃₀-C₁₀₀ substituted heteroaryl; and

wherein x and y are each independently an integer from 1 to 10.

In one embodiment, K is selected from the following K1) through K4):

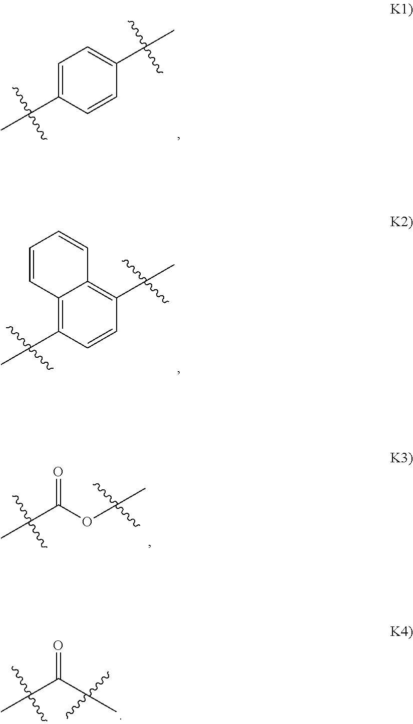

In another embodiment, L₁ is selected from the following L₁ 1) through L₁7):

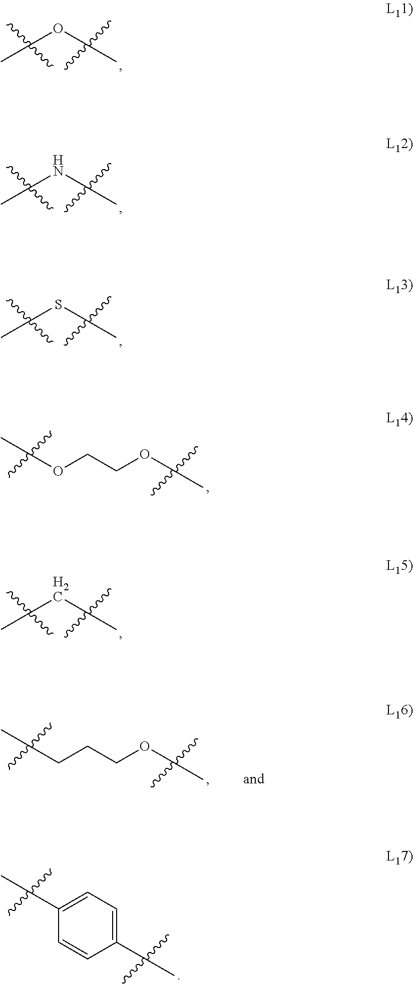

In yet another embodiment, M is selected from the following M1) through M11):

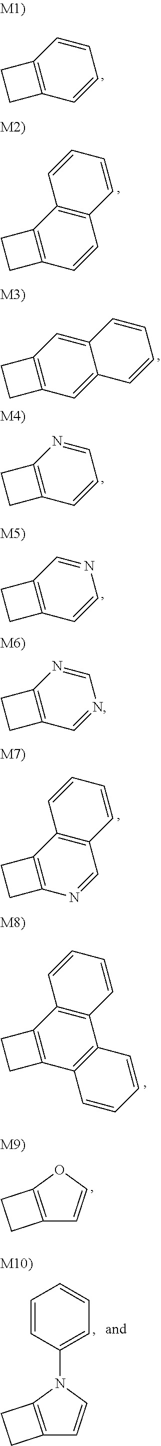

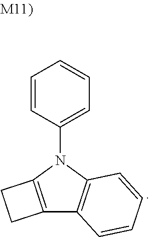 [SMILES: c1ccc(-n2c3c(c4ccccc42)CC3)cc1]

In yet another embodiment, Monomer A is selected from the following A1) through A14):

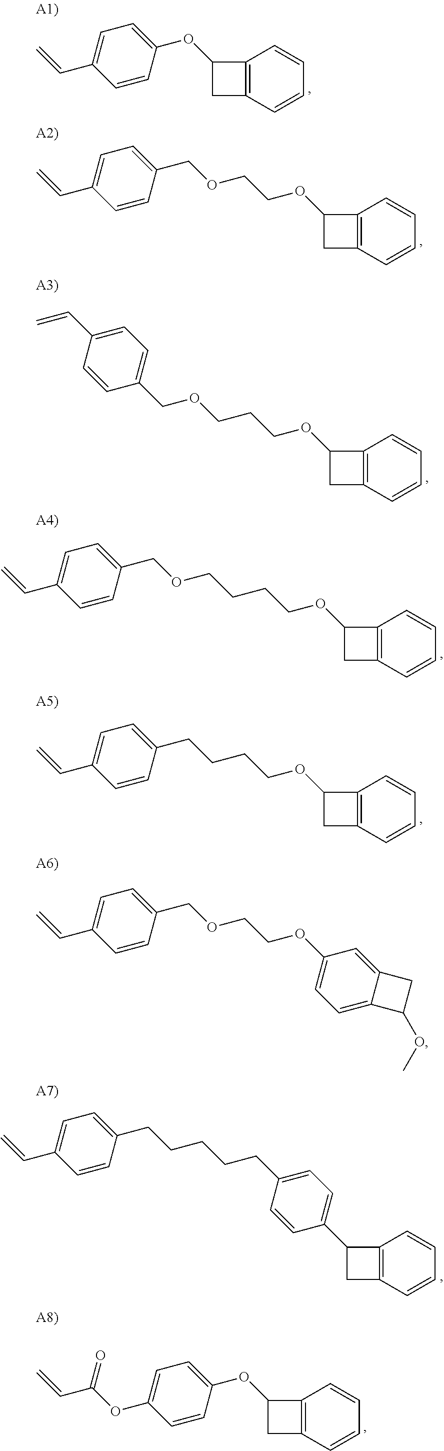

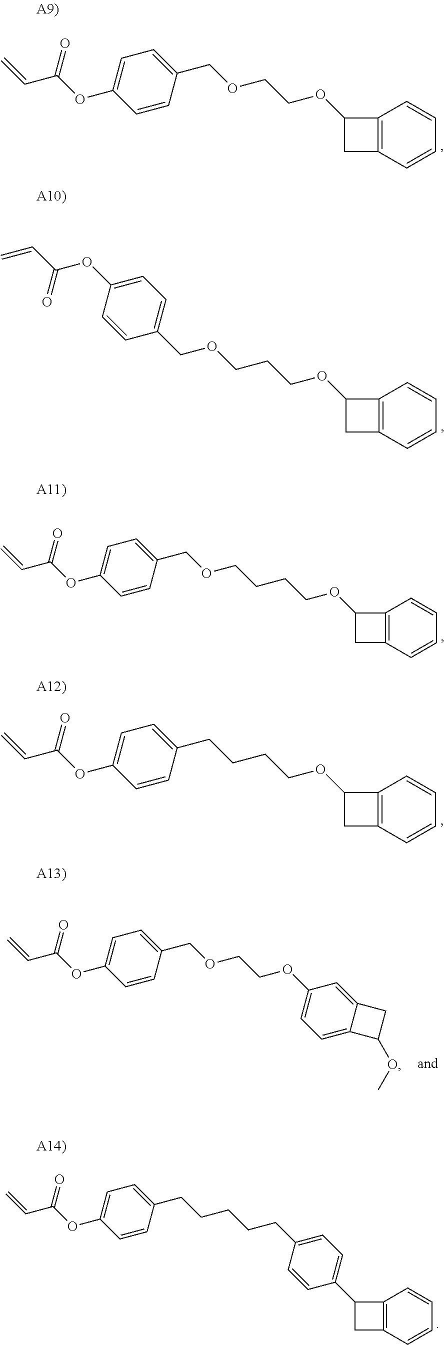 [SMILES: C=CC(=O)Oc1ccc(COCCOC2Cc3ccccc32)cc1 ; C=CC(=O)Oc1ccc(COCCCOC2Cc3ccccc32)cc1 ; C=CC(=O)Oc1ccc(COCCCCOC2Cc3ccccc32)cc1 ; C=CC(=O)Oc1ccc(CCCCOC2Cc3ccccc32)cc1 ; C=CC(=O)Oc1ccc(COCCOc2ccc3c(c2)CC3OC)cc1 ; C=CC(=O)Oc1ccc(CCCCCc2ccc(C3Cc4ccccc43)cc2)cc1]

The polymer further comprises Monomer B comprising at least one polymerizable moiety and having the following Structure B:

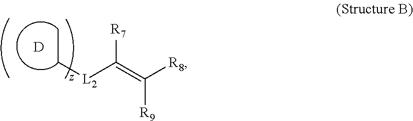

wherein D is selected from aromatic moiety, heteroaromatic moiety, C₁-C₁₀₀ hydrocarbyl, C₁-C₁₀₀ substituted hydrocarbyl, C₁-C₁₀₀ heterohydrocarbyl, and C₁-C₁₀₀ substituted heterohydrocarbyl; and

wherein L₂ may be absent, or is selected from aromatic moiety, heteroaromatic moiety, carboxy moiety, and carbonyl moiety; and

wherein R₇ through R₉ are each independently selected from hydrogen; deuterium; hydrocarbyl such as C₁-C₁₀₀ hydrocarbyl, preferably C₃-C₁₀₀ hydrocarbyl, more preferably C₁₀-C₁₀₀ hydrocarbyl, even more preferably C₂₀-C₁₀₀ hydrocarbyl, and most preferably C₃₀-C₁₀₀ hydrocarbyl; substituted hydrocarbyl such as C₁-C₁₀₀ substituted hydrocarbyl, preferably C₃-C₁₀₀ substituted hydrocarbyl, more preferably C₁₀-C₁₀₀ substituted hydrocarbyl, even more preferably C₂₀-C₁₀₀ substituted hydrocarbyl, and most preferably C₃₀-C₁₀₀ substituted hydrocarbyl; heterohydrocarbyl such as C₁-C₁₀₀ heterohydrocarbyl, preferably C₃-C₁₀₀ heterohydrocarbyl, more preferably C₁₀-C₁₀₀ heterohydrocarbyl, even more preferably C₂₀-C₁₀₀ heterohydrocarbyl, and most preferably C₃₀-C₁₀₀ heterohydrocarbyl; substituted heterohydrocarbyl such as C₁-C₁₀₀ substituted heterohydrocarbyl, preferably C₃-C₁₀₀ substituted heterohydrocarbyl, more preferably C₁₀-C₁₀₀ substituted heterohydrocarbyl, even more preferably C₂₀-C₁₀₀ substituted heterohydrocarbyl, and most preferably C₃₀-C₁₀₀ substituted heterohydrocarbyl; halogen; cyano; aryl such as C₅-C₁₀₀ aryl, preferably C₆-C₁₀₀ aryl, more preferably C₁₀-C₁₀₀ aryl, even more preferably C₂₀-C₁₀₀ aryl, and most preferably C₃₀-C₁₀₀ aryl; substituted aryl such as C₅-C₁₀₀ substituted aryl, preferably C₆-C₁₀₀ substituted aryl, more preferably C₁₀-C₁₀₀ substituted aryl, even more preferably C₂₀-C₁₀₀ substituted aryl, and most preferably C₃₀-C₁₀₀ substituted aryl; heteroaryl such as C₅-C₁₀₀ heteroaryl, preferably C₆-C₁₀ heteroaryl, more preferably C₁₀-C₁₀₀ heteroaryl, even more preferably C₂₀-C₁₀₀ heteroaryl, and most preferably C₂₀-C₁₀₀ heteroaryl; substituted heteroaryl such as C₅-C₁₀₀ substituted heteroaryl, preferably C₆-C₁₀₀ substituted heteroaryl, more preferably C₁₀-C₁₀₀ substituted heteroaryl, even more preferably C₂₀-C₁₀₀ substituted heteroaryl, and most preferably C₃₀-C₁₀₀ substituted heteroaryl; and wherein z is an integer from 1 to 10.

In one embodiment, D is selected from the following D1) through D8):

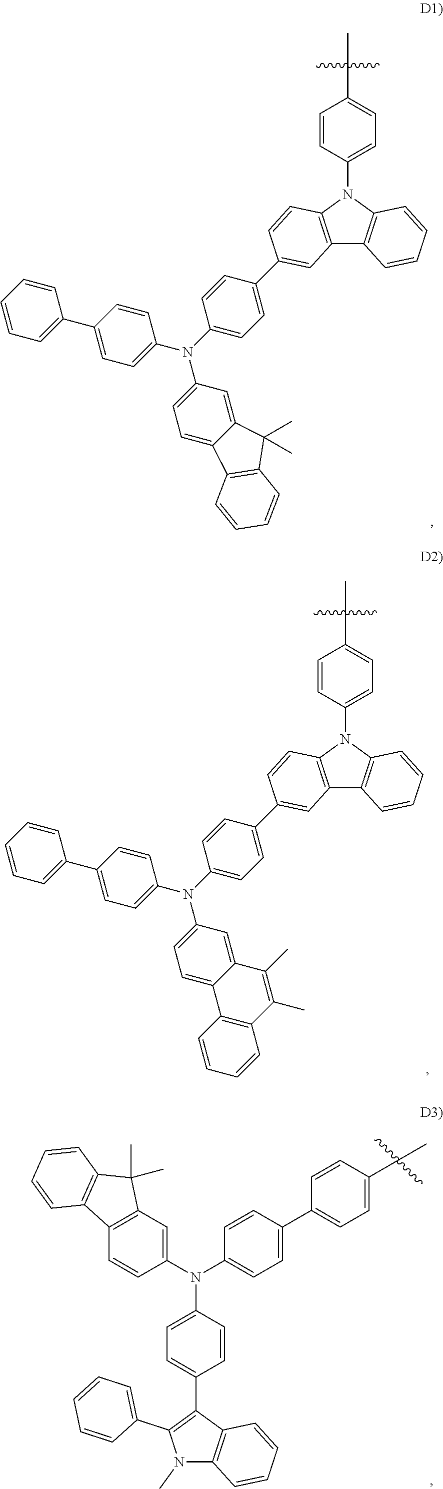 [SMILES: *c1ccc(-n2c3ccccc3c3cc(-c4ccc(N(c5ccc(-c6ccccc6)cc5)c5ccc6c(c5)C(C)(C)c5ccccc5-6)cc4)ccc32)cc1 ; *c1ccc(-n2c3ccccc3c3cc(-c4ccc(N(c5ccc(-c6ccccc6)cc5)c5ccc6c(c5)c(C)c(C)c5ccccc56)cc4)ccc32)cc1 ; *c1ccc(-c2ccc(N(c3ccc(-c4c(-c5ccccc5)n(C)c5ccccc45)cc3)c3ccc4c(c3)C(C)(C)c3ccccc3-4)cc2)cc1]

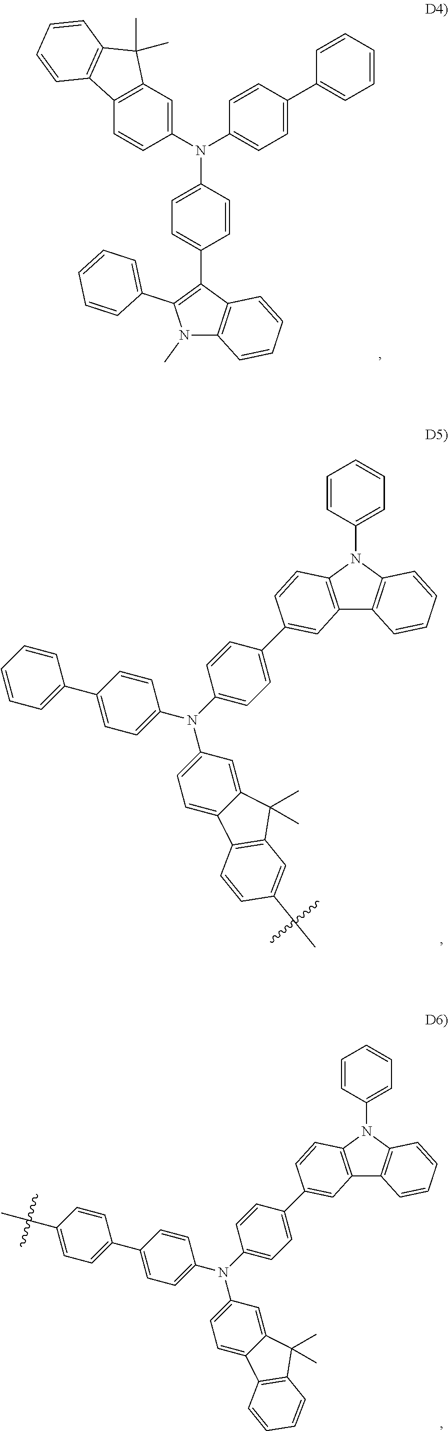 [SMILES: Cn1c(-c2ccccc2)c(-c2ccc(N(c3ccc(-c4ccccc4)cc3)c3ccc4c(c3)C(C)(C)c3ccccc3-4)cc2)c2ccccc21 ; *c1ccc2c(c1)C(C)(C)c1cc(N(c3ccc(-c4ccccc4)cc3)c3ccc(-c4ccc5c(c4)c4ccccc4n5-c4ccccc4)cc3)ccc1-2 ; *c1ccc(-c2ccc(N(c3ccc(-c4ccc5c(c4)c4ccccc4n5-c4ccccc4)cc3)c3ccc4c(c3)C(C)(C)c3ccccc3-4)cc2)cc1]

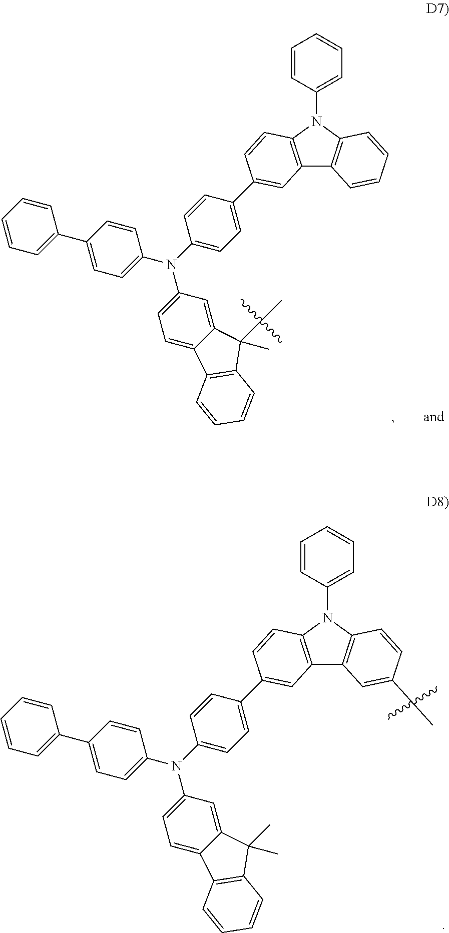 [SMILES: *C1(C)c2ccccc2-c2ccc(N(c3ccc(-c4ccccc4)cc3)c3ccc(-c4ccc5c(c4)c4ccccc4n5-c4ccccc4)cc3)cc21 ; *c1ccc2c(c1)c1cc(-c3ccc(N(c4ccc(-c5ccccc5)cc4)c4ccc5c(c4)C(C)(C)c4ccccc4-5)cc3)ccc1n2-c1ccccc1]

In another embodiment, L₂ is selected from the following L₂1) through L₂₅):

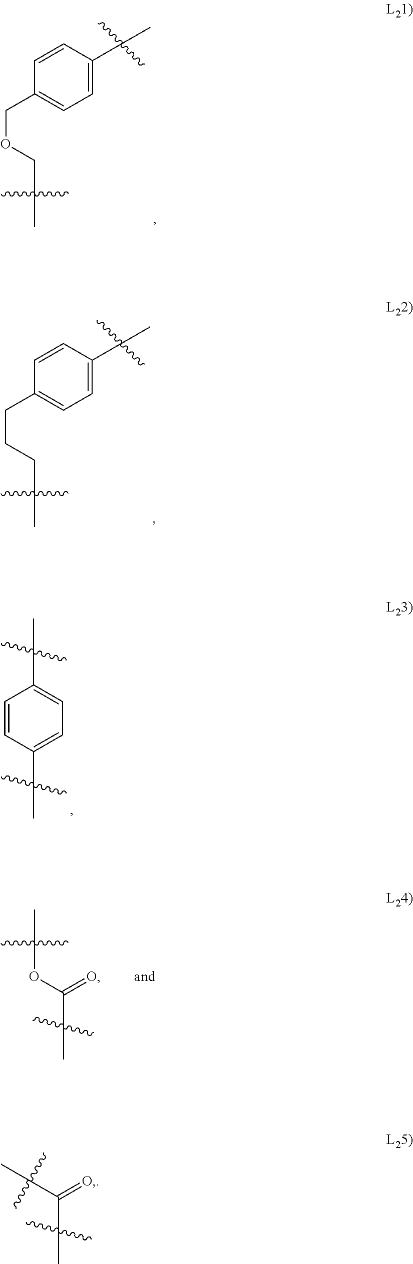

In another embodiment, Monomer B is selected from the following B1) through B18):

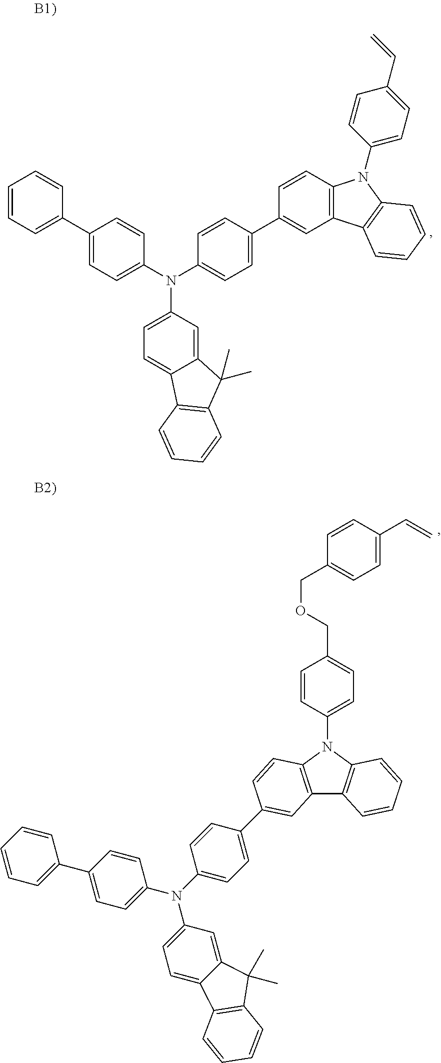 [SMILES: C=Cc1ccc(-n2c3ccccc3c3cc(-c4ccc(N(c5ccc(-c6ccccc6)cc5)c5ccc6c(c5)C(C)(C)c5ccccc5-6)cc4)ccc32)cc1 ; C=Cc1ccc(COCc2ccc(-n3c4ccccc4c4cc(-c5ccc(N(c6ccc(-c7ccccc7)cc6)c6ccc7c(c6)C(C)(C)c6ccccc6-7)cc5)ccc43)cc2)cc1]

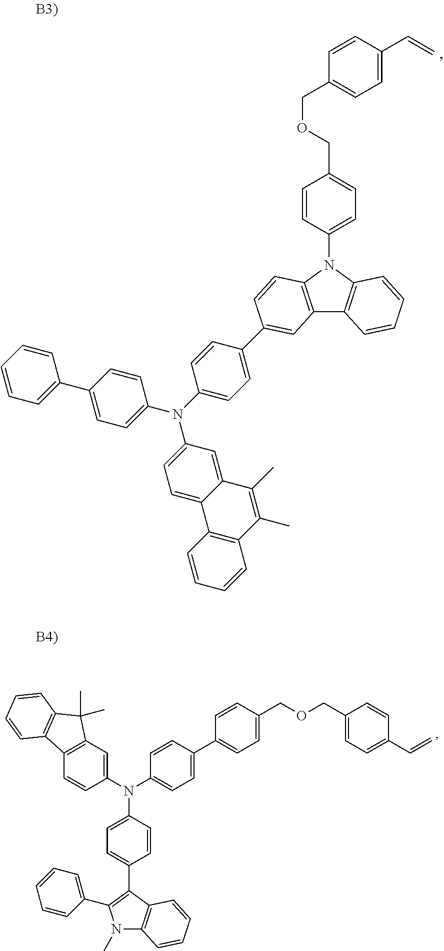 [SMILES: C=Cc1ccc(COCc2ccc(-n3c4ccccc4c4cc(-c5ccc(N(c6ccc(-c7ccccc7)cc6)c6ccc7c(c6)c(C)c(C)c6ccccc67)cc5)ccc43)cc2)cc1 ; C=Cc1ccc(COCc2ccc(-c3ccc(N(c4ccc(-c5c(-c6ccccc6)n(C)c6ccccc56)cc4)c4ccc5c(c4)C(C)(C)c4ccccc4-5)cc3)cc2)cc1]

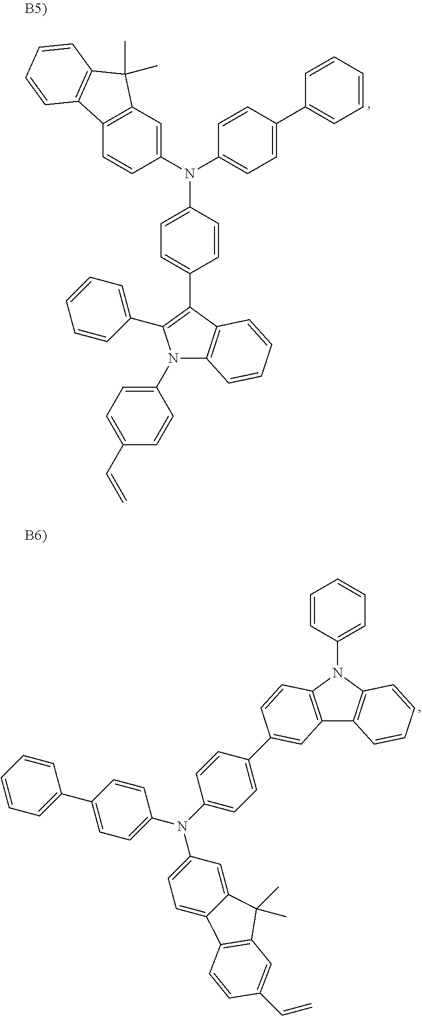 [SMILES: C=Cc1ccc(-n2c(-c3ccccc3)c(-c3ccc(N(c4ccc(-c5ccccc5)cc4)c4ccc5c(c4)C(C)(C)c4ccccc4-5)cc3)c3ccccc32)cc1 ; C=Cc1ccc2c(c1)C(C)(C)c1cc(N(c3ccc(-c4ccccc4)cc3)c3ccc(-c4ccc5c(c4)c4ccccc4n5-c4ccccc4)cc3)ccc1-2]

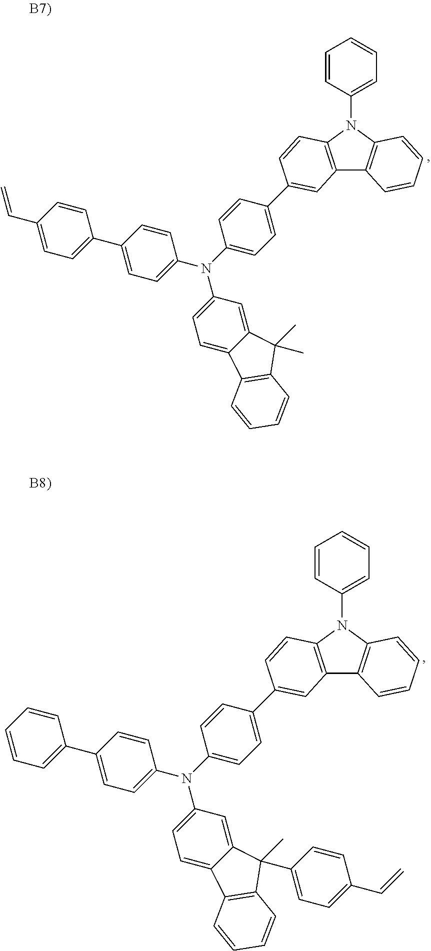 [SMILES: C=Cc1ccc(-c2ccc(N(c3ccc(-c4ccc5c(c4)c4ccccc4n5-c4ccccc4)cc3)c3ccc4c(c3)C(C)(C)c3ccccc3-4)cc2)cc1 ; C=Cc1ccc(C2(C)c3ccccc3-c3ccc(N(c4ccc(-c5ccccc5)cc4)c4ccc(-c5ccc6c(c5)c5ccccc5n6-c5ccccc5)cc4)cc32)cc1]

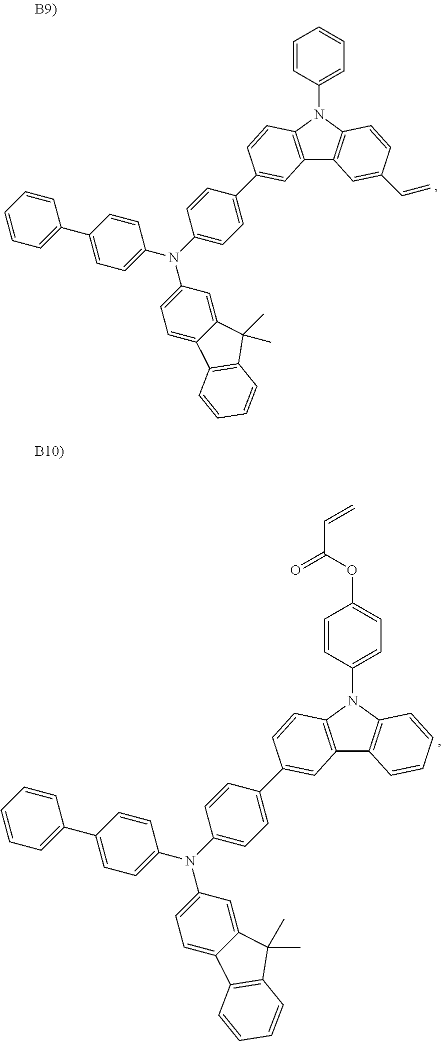 [SMILES: C=Cc1ccc2c(c1)c1cc(-c3ccc(N(c4ccc(-c5ccccc5)cc4)c4ccc5c(c4)C(C)(C)c4ccccc4-5)cc3)ccc1n2-c1ccccc1 ; C=CC(=O)Oc1ccc(-n2c3ccccc3c3cc(-c4ccc(N(c5ccc(-c6ccccc6)cc5)c5ccc6c(c5)C(C)(C)c5ccccc5-6)cc4)ccc32)cc1]

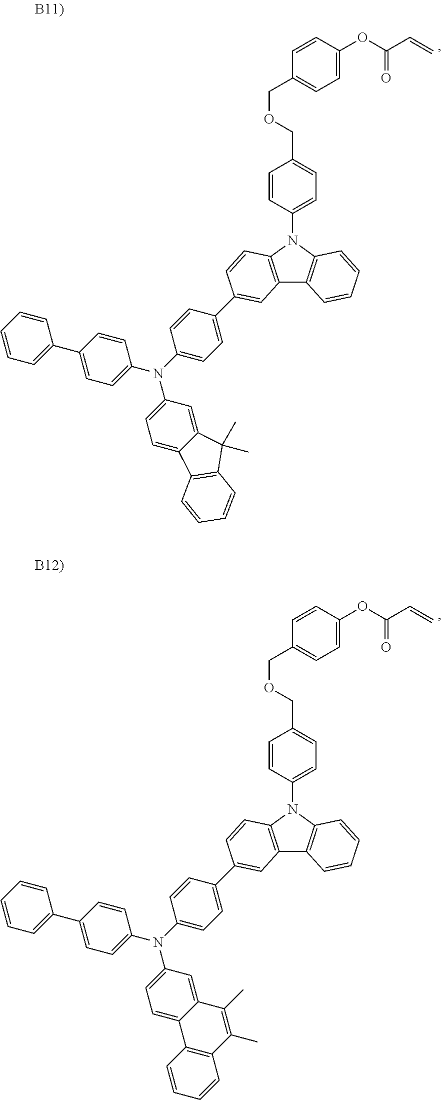 [SMILES: C=CC(=O)Oc1ccc(COCc2ccc(-n3c4ccccc4c4cc(-c5ccc(N(c6ccc(-c7ccccc7)cc6)c6ccc7c(c6)C(C)(C)c6ccccc6-7)cc5)ccc43)cc2)cc1 ; C=CC(=O)Oc1ccc(COCc2ccc(-n3c4ccccc4c4cc(-c5ccc(N(c6ccc(-c7ccccc7)cc6)c6ccc7c(c6)c(C)c(C)c6ccccc67)cc5)ccc43)cc2)cc1]

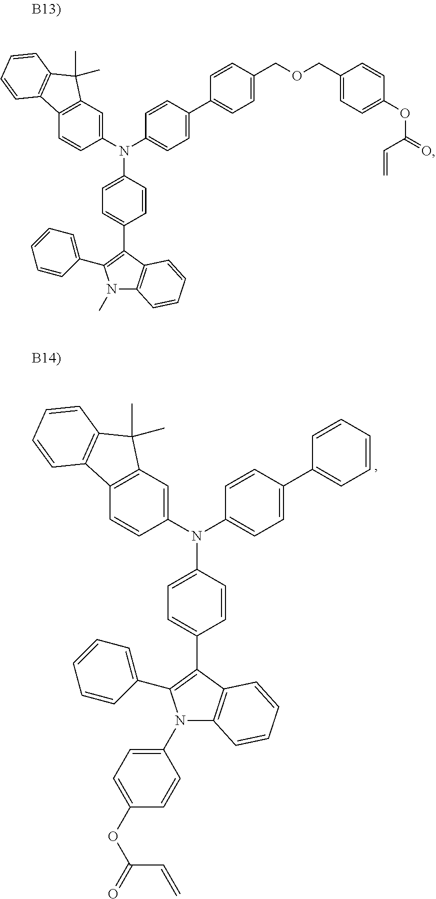 [SMILES: C=CC(=O)Oc1ccc(COCc2ccc(-c3ccc(N(c4ccc(-c5c(-c6ccccc6)n(C)c6ccccc56)cc4)c4ccc5c(c4)C(C)(C)c4ccccc4-5)cc3)cc2)cc1 ; C=CC(=O)Oc1ccc(-n2c(-c3ccccc3)c(-c3ccc(N(c4ccc(-c5ccccc5)cc4)c4ccc5c(c4)C(C)(C)c4ccccc4-5)cc3)c3ccccc32)cc1]

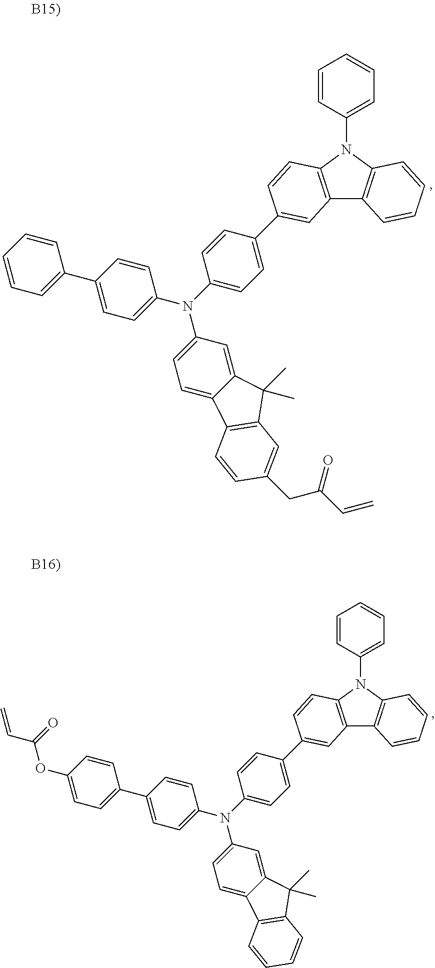 [SMILES: C=CC(=O)Cc1ccc2c(c1)C(C)(C)c1cc(N(c3ccc(-c4ccccc4)cc3)c3ccc(-c4ccc5c(c4)c4ccccc4n5-c4ccccc4)cc3)ccc1-2 ; C=CC(=O)Oc1ccc(-c2ccc(N(c3ccc(-c4ccc5c(c4)c4ccccc4n5-c4ccccc4)cc3)c3ccc4c(c3)C(C)(C)c3ccccc3-4)cc2)cc1]

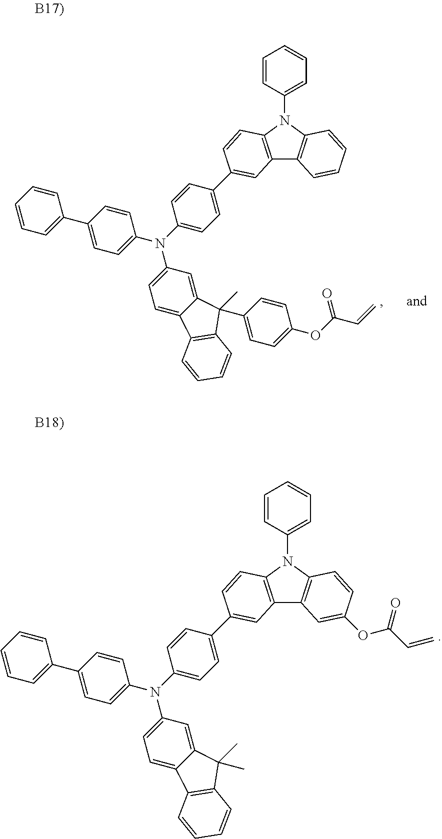 [SMILES: C=CC(=O)Oc1ccc(C2(C)c3ccccc3-c3ccc(N(c4ccc(-c5ccccc5)cc4)c4ccc(-c5ccc6c(c5)c5ccccc5n6-c5ccccc5)cc4)cc32)cc1 ; C=CC(=O)Oc1ccc2c(c1)c1cc(-c3ccc(N(c4ccc(-c5ccccc5)cc4)c4ccc5c(c4)C(C)(C)c4ccccc4-5)cc3)ccc1n2-c1ccccc1]

The monomers are made into the polymer through radical or anionic polymerization. Any ratio of two or more monomers can be used to make the polymer. In a preferred embodiment, the molar ratio of all Monomers A to all Monomers B is from 0.1:99.9 to 99.9:0.1. More preferably, it is from 5:95 to 95:5, even more preferably from 15:85 to 85:15, and most preferably from 30:70 to 70:30.

Additive

Optionally, the polymer may be blended with an additive to make the polymeric charge transfer layer composition. The additive is from 0.01% to 50%, preferably from 0.01% to 30%, and more preferably from 0.01% to 15% by dry weight based on total dry weight of the polymeric charge transfer layer composition.

The additive is an organic salt component having the following Structure C:

[(R₁₀)_{q}-E-(R₁₁)ₜ]^{⊕}[G]^{⊖} (Structure C),

wherein E is an element selected from C, Si, Ge, Sn, Pb, N, P, As, Sb, Bi, O, S, Se, Te, and I; and

wherein G is a non-coordinating anion selected from borates, hexafluoroantimonate, hexafluoroarsenate, hexafluorophosphate, tetrakis(pentafluorophenyl)borate, and tetrafluoroborate; and

wherein R₁₀ and R₁₁ are each independently selected from hydrogen; deuterium; hydrocarbyl such as C₁-C₁₀₀ hydrocarbyl, preferably C₃-C₁₀₀ hydrocarbyl, more preferably C₁₀-C₁₀₀ hydrocarbyl, even more preferably C₂₀-C₁₀₀ hydrocarbyl, and most preferably C₃₀-C₁₀₀ hydrocarbyl; substituted hydrocarbyl such as C₁-C₁₀₀ substituted hydrocarbyl, preferably C₃-C₁₀₀ substituted hydrocarbyl, more preferably C₁₀-C₁₀₀ substituted hydrocarbyl, even more preferably C₂₀-C₁₀₀ substituted hydrocarbyl, and most preferably C₃₀-C₁₀₀ substituted hydrocarbyl; heterohydrocarbyl such as C₁-C₁₀₀ heterohydrocarbyl, preferably C₃-C₁₀₀ heterohydrocarbyl, more preferably C₁₀-C₁₀₀ heterohydrocarbyl, even more preferably C₂₀-C₁₀₀ heterohydrocarbyl, and most preferably C₃₀-C₁₀₀ heterohydrocarbyl; substituted heterohydrocarbyl such as C₁-C₁₀₀ substituted heterohydrocarbyl, preferably C₃-C₁₀₀ substituted heterohydrocarbyl, more preferably C₁₀-C₁₀₀ substituted heterohydrocarbyl, even more preferably C₂₀-C₁₀₀ substituted heterohydrocarbyl, and most preferably C₃₀-C₁₀₀ substituted heterohydrocarbyl; halogen; cyano; aryl such as C₅-C₁₀₀ aryl, preferably C₆-C₁₀₀ aryl, more preferably C₁₀-C₁₀₀ aryl, even more preferably C₂₀-C₁₀₀ aryl, and most preferably C₃₀-C₁₀₀ aryl; substituted aryl such as C₅-C₁₀₀ substituted aryl, preferably C₆-C₁₀₀ substituted aryl, more preferably C₁₀-C₁₀₀ substituted aryl, even more preferably C₂₀-C₁₀₀ substituted aryl, and most preferably C₃₀-C₁₀₀ substituted aryl; heteroaryl such as C₅-C₁₀₀ heteroaryl, preferably C₆-C₁₀ heteroaryl, more preferably C₁₀-C₁₀₀ heteroaryl, even more preferably C₂₀-C₁₀₀ heteroaryl, and most preferably C₂₀-C₁₀₀ heteroaryl; substituted heteroaryl such as C₅-C₁₀₀ substituted heteroaryl, preferably C₆-C₁₀₀ substituted heteroaryl, more preferably C₁₀-C₁₀₀ substituted heteroaryl, even more preferably C₂₀-C₁₀₀ substituted heteroaryl, and most preferably C₃₀-C₁₀₀ substituted heteroaryl; and

wherein q is an integer from 1 to v+1, and t is an integer from 0 to v−1; and

wherein v is the valence of the element E, and q+t=v+1.

In one embodiment, the additive is selected from the following C1) through C10):

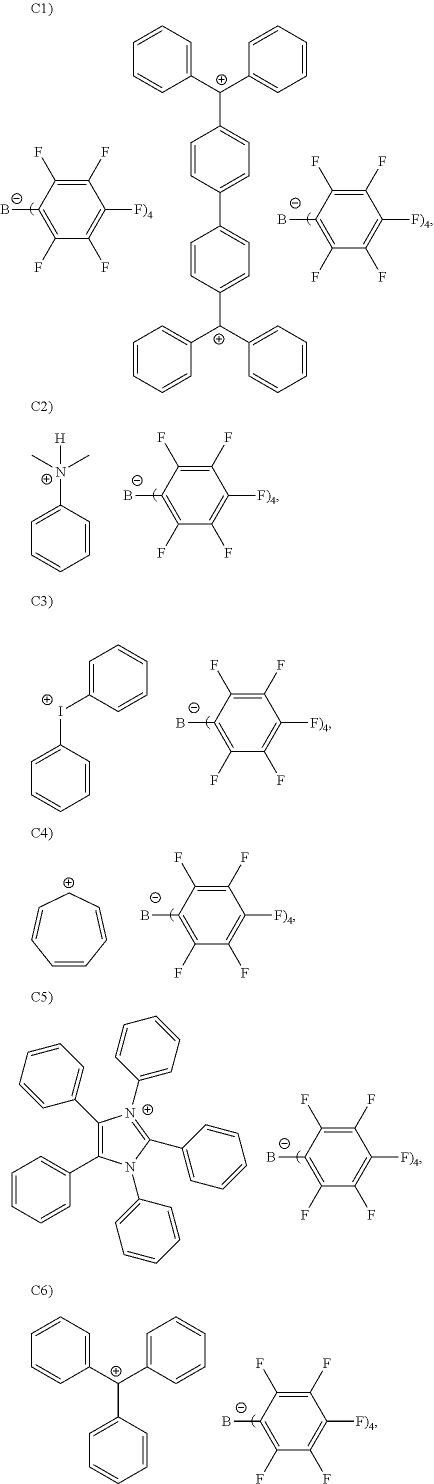

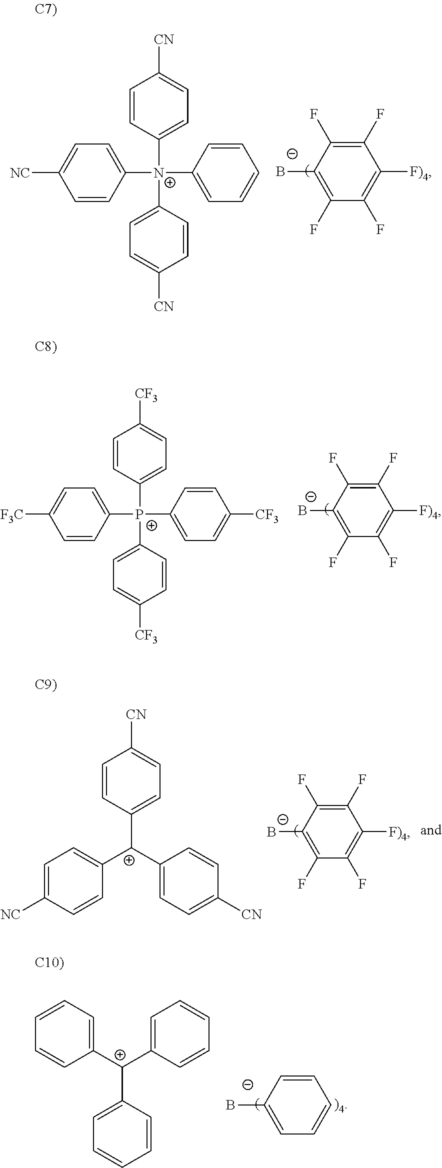

Organic Electronic Device

The present invention provides a method of making an organic electronic device. The method comprises providing the polymeric charge transfer layer solution of the present invention, and dissolving or dispersing the polymeric charge transfer layer solution in any of the organic solvents known or proposed to be used in the fabrication of an organic electronic device by solution process. Such organic solvents include including tetrahydrofuran (THF), cyclohexanone, chloroform, 1,4-dioxane, acetonitrile, ethyl acetate, tetralin, chlorobenzene, toluene, xylene, anisole, mesitylene, tetralone, and any combination thereof. The polymeric charge transfer layer solution was filtered through a membrane or a filter to remove particles larger than 50 nm.

The polymeric charge transfer layer solution is then deposited over a first electrode, which may be an anode or cathode. The deposition may be performed by any of various types of solution processing techniques known or proposed to be used for fabricating light emitting devices. For example, the polymeric charge transfer layer solution can be deposited using a printing process, such as inkjet printing, nozzle printing, offset printing, transfer printing, or screen printing; or for example, using a coating process, such as spray coating, spin coating, or dip coating. After deposition of the solution, the solvent is removed, which may be performed by using conventional method such as vacuum drying or heating.

The polymeric charge transfer layer solution is further cross-linked to form the layer. Cross-linking may be performed by exposing the layer solution to heat and/or actinic radiation, including UV light, gamma rays, or x-rays. Cross-linking may be carried out in the presence of an initiator that decomposed under heat or irradiation to produce free radicals or ions that initiate the cross-linking reaction. The cross-linking may be performed in-situ during the fabrication of a device. After cross-linking, the polymeric charge transfer layer made thereof is preferably free of residual moieties which are reactive or decomposable with exposure to light, positive charges, negative charges or excitons.

The process of solution deposition and cross-linking can be repeated to create multiple layers.

The organic light emitting device of the present invention comprises a first conductive layer, an electron transport layer (ETL), an emissive layer (EML), and one or more hole transport layers (HTL) and a second conductive layer. The hole transport layer, as the typical polymeric charge transfer layer, is prepared according to the above process. The first conductive layer is used as an anode and in general is a transparent conducting oxide, for example, fluorine-doped tin oxide, antimony-doped tin oxide, zinc oxide, aluminum-doped zinc oxide, indium tin oxide, metal nitride, metal selenide and metal sulfide. The second conductive layer is a cathode and comprises a conductive material. It is preferred that the material has a good thin film-forming property to ensure sufficient contact between the second conductive layer and hole transport layer to promote the electron injection under low voltage and provide better stability. For example, the material of the cathode can be a metal such as aluminum and calcium, a metal alloy such as magnesium/silver and aluminum/lithium, and any combination thereof. Moreover, an extremely thin film of lithium fluoride may be optionally placed between the cathode and the emitting layer. Lithium fluoride can effectively reduce the energy barrier of injecting electrons from the cathode to the emitting layer. In addition, the emitting layer plays a very important role in the whole structure of the light emitting device. In addition to determining the color of the device, the emitting layer also has an important impact on the luminance efficiency in a whole. Common luminescent materials can be classified as fluorescence and phosphorescence depending on the light emitting mechanism.

### Definitions

The term “organic electronic device,” refers to a device that carries out an electrical operation with the presence of organic materials. Specific example includes organic light emitting devices, organic solar cells, organic memory devices, organic sensors, organic thin film transistors, and power generation and storage devices such as organic batteries, fuel cells, and organic supercapacitors.

The term “organic light emitting device,” refers to a device that emits light when an electrical current is applied across two electrodes. Specific example includes light emitting diodes.

The term “polymeric charge transfer layer,” refers to a polymeric material that can transport charge carrying moieties, either holes or electrons. Specific example includes hole transport layer.

The term “aromatic moiety,” refers to an organic moiety derived from aromatic hydrocarbon by deleting at least one hydrogen atom therefrom. An aromatic moiety may be a monocyclic and/or fused ring system, each ring of which suitably contains from 3 to 7, preferably from 5 or 6 atoms. Structures wherein two or more aromatic moieties are combined through single bond(s) are also included. Specific examples include phenyl, naphthyl, biphenyl, anthryl, indenyl, fluorenyl, benzofluorenyl, phenanthryl, triphenylenyl, pyrenyl, perylenyl, chrysenyl, naphtacenyl, and fluoranthenyl. The naphthyl may be 1-naphthyl or 2-naphthyl, the anthryl may be 1-anthryl, 2-anthryl or 9-anthryl, and the fluorenyl may be any one of 1-fluorenyl, 2-fluorenyl, 3-fluorenyl, 4-fluorenyl and 9-fluorenyl.

The term “heteroaromatic moiety,” refers to an aromatic moiety, in which at least one carbon atom or CH group or CH₂ group is substituted with a heteroatom or a chemical group containing at least one heteroatom. The heteroaromatic moiety may be a 5- or 6-membered monocyclic heteroaryl, or a polycyclic heteroaryl which is fused with one or more benzene ring(s), and may be partially saturated. The structures having one or more heteroaromatic moieties bonded through a single bond are also included. Specific examples include monocyclic heteroaryl groups, such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; polycyclic heteroaryl groups, such as benzofuranyl, fluoreno[4, 3-b]benzofuranyl, benzothiophenyl, fluoreno[4, 3-b]benzothiophenyl, isobenzofuranyl, benzimidazolyl, benzothiazolyl, benzisothiazolyl, benzisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenanthridinyl and benzodioxolyl.

The term “hydrocarbyl,” refers to a chemical group containing only hydrogen and carbon atoms.

The term “substituted hydrocarbyl,” refers to a hydrocarbyl in which at least one hydrogen atom is substituted with a heteroatom or a chemical group containing at least one heteroatom.

The term “heterohydrocarbyl,” refers to a chemical group containing hydrogen and carbon atoms, and wherein at least one carbon atom or CH group or CH₂ group is substituted with a heteroatom or a chemical group containing at least one heteroatom.

The term “substituted heterohydrocarbyl,” refers to a heterohydrocarbyl in which at least one hydrogen atom is substituted with a heteroatom or a chemical group containing at least one heteroatom.

The term “aryl,” refers to an organic radical derived from aromatic hydrocarbon by deleting one hydrogen atom therefrom. An aryl group may be a monocyclic and/or fused ring system, each ring of which suitably contains from 4 to 7, preferably from 5 or 6 atoms. Structures wherein two or more aryl groups are combined through single bond(s) are also included. Specific examples include phenyl, naphthyl, biphenyl, anthryl, indenyl, fluorenyl, benzofluorenyl, phenanthryl, triphenylenyl, pyrenyl, perylenyl, chrysenyl, naphtacenyl, and fluoranthenyl. The naphthyl may be 1-naphthyl or 2-naphthyl, the anthryl may be 1-anthryl, 2-anthryl or 9-anthryl, and the fluorenyl may be any one of 1-fluorenyl, 2-fluorenyl, 3-fluorenyl, 4-fluorenyl and 9-fluorenyl.

The term “substituted aryl,” refers to an aryl in which at least one hydrogen atom is substituted with a heteroatom or a chemical group containing at least one heteroatom.

The term “heteroaryl,” refers to an aryl group, in which at least one carbon atom or CH group or CH₂ group is substituted with a heteroatom or a chemical group containing at least one heteroatom. The heteroaryl may be a 5- or 6-membered monocyclic heteroaryl or a polycyclic heteroaryl which is fused with one or more benzene ring(s), and may be partially saturated. The structures having one or more heteroaryl group(s) bonded through a single bond are also included. The heteroaryl groups may include divalent aryl groups of which the heteroatoms are oxidized or quarternized to form N-oxides, quaternary salts, or the like. Specific examples include, but are not limited to, monocyclic heteroaryl groups, such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl; polycyclic heteroaryl groups, such as benzofuranyl, fluoreno[4, 3-b]benzofuranyl, benzothiophenyl, fluoreno[4, 3-b]benzothiophenyl, isobenzofuranyl, benzimidazolyl, benzothiazolyl, benzisothiazolyl, benzisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenanthridinyl and benzodioxolyl; and corresponding N-oxides (for example, pyridyl N-oxide, quinolyl N-oxide) and quaternary salts thereof.

The term “substituted heteroaryl,” refers to a heteroaryl in which at least one hydrogen atom is substituted with a heteroatom or a chemical group containing at least one heteroatom.

Heteroatoms include O, N, P, P(═O), Si, B, F, Cl, Br, I, D and S.

The term “polymer,” refers to a polymeric compound prepared by polymerizing monomers, whether of the same or a different type. The generic term polymer thus embraces the term homopolymer (employed to refer to polymers prepared from only one type of monomer, with the understanding that trace amounts of impurities can be incorporated into and/or within the polymer structure), and the term copolymer as defined hereinafter.

The term “copolymer,” refers to polymers prepared by the polymerization of at least two different types of monomers.

### EXAMPLES

### I. Reagents and Test Methods

All solvents and reagents were obtained from commercial vendors, for example, Sigma-Aldrich, TCI, and Alfa Aesar, were used in the highest available purities, and/or when necessary, recrystallized before use. Dry solvents were obtained from in-house purification/dispensing system (hexane, toluene, and tetrahydrofuran), or purchased from Sigma-Aldrich. All experiments involving “water sensitive compounds” were conducted in “oven dried” glassware, under nitrogen atmosphere, or in a glovebox.

¹H-NMR-spectra (500 MHz or 400 MHz) were obtained on a Varian VNMRS-500 or VNMRS-400 spectrometer at 25° C., unless otherwise noted. The chemical shifts were referenced as follows: CHCl₃(5=7.26) in CDCl₃. ¹³C-NMR-spectra (125 MHz or 100 MHz) were obtained on a Varian VNMRS-500 or VNMRS-400 spectrometer at 25° C., unless otherwise noted. The chemical shifts were referenced as follows: CDCl₃ (δ=77.0).

Routine liquid chromatography/mass spectrometry (LC/MS) studies were carried out as follows. One microliter aliquots of the sample, as “1 mg/ml solution in tetrahydrofuran (THF),” were injected on an Agilent 1200SL binary liquid chromatography (LC), coupled to an Agilent 6520 quadruple time-of-flight (Q-TOF) MS system, via a dual electrospray interface (ESI), operating in the PI mode. The following analysis conditions were used: Column: Agilent Eclipse XDB-C18, 4.6*50 mm, 1.7 um; Column oven temperature: 30° C.; Solvent A: THF; Solvent B: 0.1% formic acid in water/Acetonitrile (v/v, 95/5); Gradient: 40-80% Solvent A in 0-6 min, and held for 9 min; Flow 0.3 mL/min; UV detector: diode array, 254 nm; MS condition: Capillary Voltage: 3900 kV (Neg), 3500 kV (Pos); Mode: Neg and Pos; Scan: 100-2000 amu; Rate: is/scan; Desolvation temperature: 300° C.

Gel permeation chromatography (GPC) studies were carried out as follows. 2 mg of B-staged HTL polymer was dissolved in 1 mL THF. The solution was filtrated through a 0.20 μm polytetrafluoroethylene (PTFE) syringe filter and 50 μl of the filtrate was injected to the GPC system. The following analysis conditions were used: Pump: Waters™ e2695 Separations Modules at a nominal flow rate of 1.0 mL/min; Eluent: Fisher Scientific HPLC grade THF (unstabilized); Injector: Waters e2695 Separations Modules; Columns: two 5 μm mixed-C columns from Polymer Laboratories Inc., held at 40° C.; Detector: Shodex RI-201 Differential Refractive Index (DRI) Detector; Calibration: 17 polystyrene standard materials from Polymer Laboratories Inc., fit to a 3rd order polynomial curve over the range of 3,742 kg/mol to 0.58 kg/mol.

### II. Examples

### 1. Synthesis of 7-(2-((4-vinylbenzylloxylethoxy)bicyclo[4.2.0]octa-1,3,5-triene (Monomer A2)

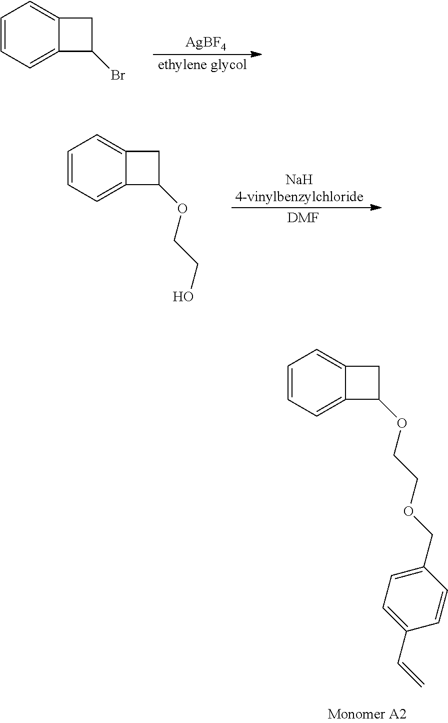

7-bromobicyclo[4.2.0]octa-1,3,5-triene (10.0 g, 54.6 mmol) and ethylene glycol (100 mL) were added to a 250 mL round-bottom flask. The biphasic mixture was cooled to 0° C. followed by the slow addition of solid silver(I) tetrafluoroborate (11.7 g, 60.1 mmol) to maintain a temperature of about 30° C. After addition, the reaction mixture was stirred at 50° C. for 3 hrs. Once cooled down to room temperature, 200 ml water and 400 ml ether were added. The resulting mixture was filtered through celite. The organic layer was washed three times with each 300 ml water and then dried over Na₂SO₄ and concentrated to give an oil. The oil was purified by column chromatography on silica gel using 5% ethyl acetate in hexanes (4.66 g). The product had the following characteristics: ¹H-NMR (400 MHz, CDCl₃): δ 7.36-7.27 (m, 1H), 7.27-7.20 (m, 2H), 7.15 (dp, J=7.2, 1.0 Hz, 1H), 5.20-5.03 (m, 1H), 3.90-3.61 (m, 4H), 3.56-3.39 (m, 1H), 3.21- (m, 1H), 2.23-1.98 (br s, 1H). ¹³C-NMR (101 MHz, CDCl₃): δ 145.64, 142.42, 129.49, 127.10, 123.54, 122.70, 76.96, 69.75, 61.91, 38.53.

In an inert atmosphere, a 100 mL round-bottom flask was charged with 2-(bicyclo[4.2.0]octa-1,3,5-trien-7-yloxy)ethan-1-ol (3.0 g, 18.3 mmol) and 50 mL DMF. Solid NaH (0.658 g, 27.4 mmol) was added portion wise and the mixture was stirred at room temperature for 1 hour. A 3 mL DMF solution of 4-vinyl benzylchloride (4.18 g, 27.4 mmol) was added dropwise and the mixture was heated to 60° C. After stirring the mixture overnight, the reaction was cooled to room temperature and poured into 100 mL water. The product was extracted with diethyl ether (100 mL) several times and the organic fraction was combined and dried with MgSO₄. The solvent was removed and the product was purified by column chromatography on silica gel with 5% ethyl acetate in hexanes (2.09 g). The product had the following characteristics: 41-NMR (400 MHz, CDCl₃): δ 7.36-7.27 (m, 1H), 7.27-7.20 (m, 2H), 7.15 (dp, J=7.2, 1.0 Hz, 1H), 5.10 (dd, J=4.4, 2.0 Hz, 1H), 3.90-3.61 (m, 4H), 3.56-3.39 (m, 1H), 3.21-3.03 (m, 1H), 2.23-1.98 (m, 1H). ¹³C-NMR (101 MHz, CDCl₃): δ 145.64, 142.42, 129.49, 127.10, 123.54, 122.70, 76.96, 69.75, 61.91, 38.53.

### 2. Synthesis of 4-(3-(4-([1,1′-biphenyl]-4-yl(9,9-dimethyl-9H-fluoren-2-yl)amino)phenyl)-9H-carbazol-9-yl)benzaldehyde (Formula 1)

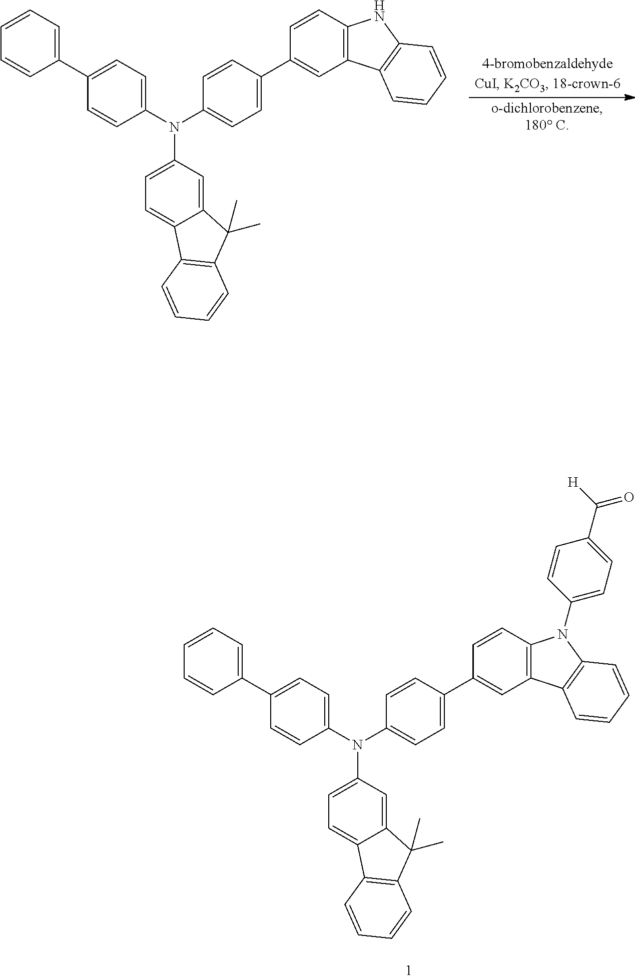 [SMILES: CC1(C)c2ccccc2-c2ccc(N(c3ccc(-c4ccccc4)cc3)c3ccc(-c4ccc5[nH]c6ccccc6c5c4)cc3)cc21 ; CC1(C)c2ccccc2-c2ccc(N(c3ccc(-c4ccccc4)cc3)c3ccc(-c4ccc5c(c4)c4ccccc4n5-c4ccc(C=O)cc4)cc3)cc21]

A round-bottom flask was charged with N-(4-(9H-carbazol-3-yephenyl)-N-([1,1′-biphenyl]-4-yl)-9,9-dimethyl-9H-fluoren-2-amine (2.00 g, 3.318 mmol, 1.0 equiv), 4-bromobenzaldehyde (0.737 g, 3.982 mmol, 1.2 equiv), CuI (0.126 g, 0.664 mmol, 0.2 equiv), potassium carbonate (1.376 g, 9.954 mmol, 3.0 equiv), and 18-crown-6 (86 mg, 10 mol %). The flask was flushed with nitrogen and connected to a reflux condenser. 10.0 mL dry, degassed 1,2-dichlorobenzene was added, and the mixture was refluxed for 48 hours. The cooled solution was quenched with sat. aq. NH₄C₁, and extracted with dichloromethane. Combined organic fractions were dried, and solvent was removed by distillation. The crude residue was purified by chromatography on silica gel (hexane/chloroform gradient), and gave a bright yellow solid product (2.04 g). The product had the following characteristics: ¹H-NMR (500 MHz, CDCl₃): δ 10.13 (s, 1H), 8.37 (d, J=2.0 Hz, 1H), 8.20 (dd, J=7.7, 1.0 Hz, 1H), 8.16 (d, J=8.2 Hz, 2H), 7.83 (d, J=8.1 Hz, 2H), 7.73-7.59 (m, 7H), 7.59-7.50 (m, 4H), 7.50-7.39 (m, 4H), 7.39-7.24 (m, 10H), 7.19-7.12 (m, 1H), 1.47 (s, 6H). ¹³C-NMR (126 MHz, CDCl₃): δ 190.95, 155.17, 153.57, 147.21, 146.98, 146.69, 143.38, 140.60, 140.48, 139.28, 138.93, 135.90, 135.18, 134.64, 134.46, 133.88, 131.43, 128.76, 127.97, 127.81, 126.99, 126.84, 126.73, 126.65, 126.54, 126.47, 125.44, 124.56, 124.44, 124.12, 123.98, 123.63, 122.49, 120.96, 120.70, 120.57, 119.47, 118.92, 118.48, 110.05, 109.92, 46.90, 27.13.

### 3. Synthesis of N-([1,1′-biphenyl]-4-yl)-9,9-dimethyl-N-(4-(9-(4-vinylphenyl)-9H-carbazol-3-yl)phenyl)-9H-fluoren-2-amine (Monomer B1)

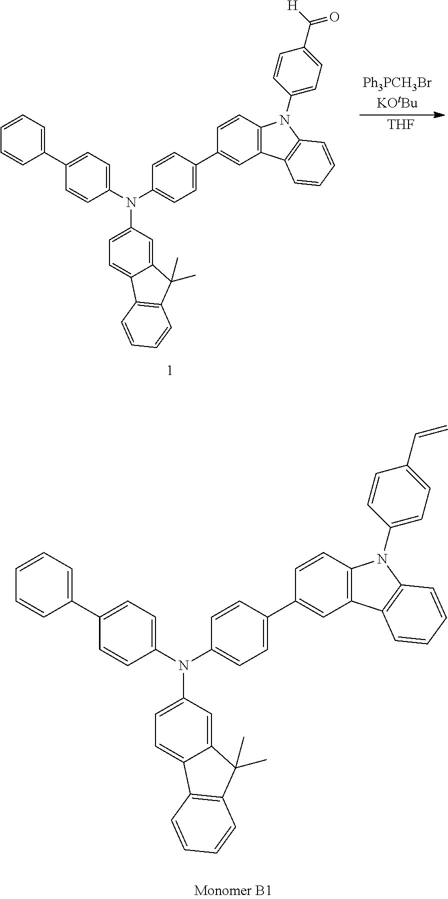 [SMILES: CC1(C)c2ccccc2-c2ccc(N(c3ccc(-c4ccccc4)cc3)c3ccc(-c4ccc5c(c4)c4ccccc4n5-c4ccc(C=O)cc4)cc3)cc21 ; C=Cc1ccc(-n2c3ccccc3c3cc(-c4ccc(N(c5ccc(-c6ccccc6)cc5)c5ccc6c(c5)C(C)(C)c5ccccc5-6)cc4)ccc32)cc1 ; "THF"]

A round-bottom flask was charged with methyl triphenylphosphonium bromide (1.87 g, 5.296 mmol, 2.00 equiv) and 8.0 mL dry THF under a blanket of nitrogen. The suspension was stirred vigorously and potassium tert-butoxide (0.743 g, 6.619 mmol, 2.50 equiv) was added slowly, and the solution was allowed to stir for 10 minutes. A solution of Formula 1 (1.85 g, 2.619 mmol, 1.0 equiv) in 8 mL THF was added to the yellow ylide slurry. After stirring at ambient temperature for 16 hours, the reaction was quenched with water and extracted with chloroform. Combined organic fractions were washed with brine and adsorbed onto silica. The compound was purified by chromatography (hexane/chloroform gradient), and gave a white solid product (4.34 g). The material's purity could be slightly enhanced by precipitation from chloroform with methanol. The product had the following characteristics: ¹H-NMR (400 MHz, CDCl₃): δ 8.37 (d, J=1.7 Hz, 1H), 8.20 (dd, J=7.8, 1.0 Hz, 1H), 7.76-7.60 (m, 9H), 7.60-7.51 (m, 4H), 7.51-7.39 (m, 6H), 7.39-7.28 (m, 8H), 7.16 (dd, J=8.1, 2.1 Hz, 1H), 6.85 (dd, J=17.6, 10.9 Hz, 1H), 5.87 (d, J=17.6 Hz, 1H), 5.38 (d, J=10.9 Hz, 1H), 1.47 (s, 6H). ¹³C-NMR: (126 MHz, CDCl₃): δ 155.15, 153.58, 147.27, 147.04, 146.46, 141.22, 140.64, 140.06, 138.96, 137.05, 136.74, 136.36, 135.96, 135.08, 134.37, 133.01, 128.74, 127.97, 127.78, 127.61, 126.99, 126.97, 126.80, 126.64, 126.49, 126.11, 125.16, 124.54, 123.96, 123.90, 123.56, 123.54, 122.47, 120.67, 120.36, 120.09, 119.45, 118.85, 118.34, 114.76, 110.05, 109.94, 46.89, 27.12.

### 4. Synthesis of (4-(3-(4-([1,1′-biphenyl]-4-yl(9,9-dimethyl-9H-fluoren-2-yl)amino)phenyl)-9H-carbazol-9-yllphenyllmethanol (Formula 2)

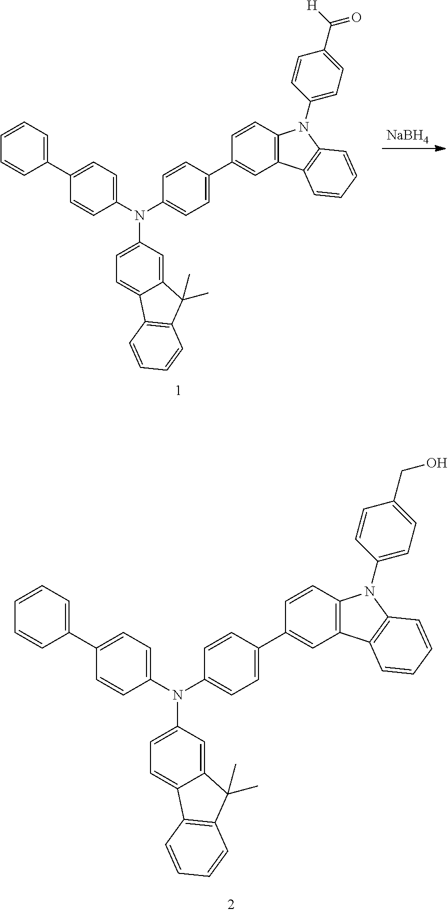 [SMILES: CC1(C)c2ccccc2-c2ccc(N(c3ccc(-c4ccccc4)cc3)c3ccc(-c4ccc5c(c4)c4ccccc4n5-c4ccc(C=O)cc4)cc3)cc21 ; "NaBH4" ; CC1(C)c2ccccc2-c2ccc(N(c3ccc(-c4ccccc4)cc3)c3ccc(-c4ccc5c(c4)c4ccccc4n5-c4ccc(CO)cc4)cc3)cc21]

A round-bottom flask was charged with Formula 1 (4.36 g, 6.17 mmol, 1.00 equiv) under a blanket of nitrogen. The material was dissolved in 40 mL 1:1 THF:EtOH. borohydride (0.280 g, 7.41 mmol, 1.20 equiv) was added in portions and the material was stirred for 3 hours. The reaction mixture was cautiously quenched with 1M HCl, and the product was extracted with portions of dichloromethane. Combined organic fractions were washed with sat. aq. sodium bicarbonate, dried with MgSO₄ and concentrated to a crude residue. The material was purified by chromatography (hexane/dichloromethane gradient), and gave a white solid product (3.79 g). The product had the following characteristics: 41-NMR (500 MHz, CDCl₃): δ 8.35 (s, 1H), 8.19 (dt, J=7.8, 1.1 Hz, 1H), 7.73-7.56 (m, 11H), 7.57-7.48 (m, 2H), 7.48-7.37 (m, 6H), 7.36-7.23 (m, 9H), 7.14 (s, 1H), 4.84 (s, 2H), 1.45 (s, 6H). ¹³C-NMR (126 MHz, CDCl₃): δ 155.13, 153.56, 147.24, 147.02, 146.44, 141.27, 140.60, 140.11, 140.07, 138.94, 136.99, 136.33, 135.06, 134.35, 132.96, 128.73, 128.44, 127.96, 127.76, 127.09, 126.96, 126.79, 126.62, 126.48, 126.10, 125.15, 124.52, 123.90, 123.54, 123.49, 122.46, 120.66, 120.36, 120.06, 119.43, 118.82, 118.33, 109.95, 109.85, 64.86, 46.87, 27.11.

### 5. Synthesis of N-([1,1′-biphenyl]-4-yl)-9,9-dimethyl-N-(4-(9-(4-(((4-vinylbenzylloxylmethyllphenyl)-9H-carbazol-3-yl)phenyl)-9H-fluoren-2-amine (Monomer B2)

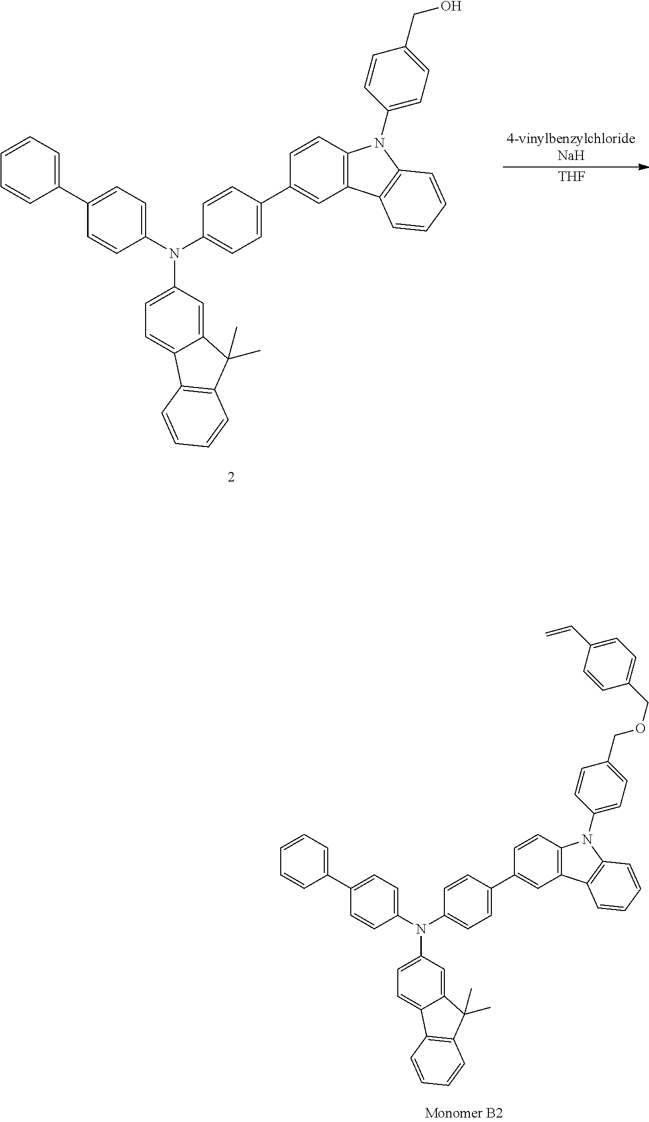

In a nitrogen-filled glovebox, a 100 mL round-bottom flask was charged with Formula 2 (4.40 g, 6.21 mmol, 1.00 equiv) and 35 mL THF. Sodium hydride (0.224 g, 9.32 mmol, 1.50 equiv) was added in portions, and the mixture was stirred for 30 minutes. A reflux condenser was attached, the unit was sealed and removed from the glovebox. 4-vinylbenzyl chloride (1.05 mL, 7.45 mmol, 1.20 equiv) was injected, and the mixture was refluxed until consumption of starting material. The reaction mixture was cooled (iced bath) and cautiously quenched with isopropanol. Sat. aq. NH₄Cl was added, and the product was extracted with ethyl acetate. Combined organic fractions were washed with brine, dried with MgSO₄, filtered, concentrated, and purified by chromatography on silica. The product had the following characteristics: ¹H-NMR (400 MHz, CDCl₃): δ 8.35 (s, 1H), 8.18 (dt, J=7.8, 1.0 Hz, 1H), 7.74-7.47 (m, 14H), 7.47-7.35 (m, 11H), 7.35-7.23 (m, 9H), 7.14 (s, 1H), 6.73 (dd, J=17.6, 10.9 Hz, 1H), 5.76 (dd, J=17.6, 0.9 Hz, 1H), 5.25 (dd, J=10.9, 0.9 Hz, 1H), 4.65 (s, 4H), 1.45 (s, 6H). ¹³C-NMR (101 MHz, CDCl₃): δ 155.13, 153.56, 147.25, 147.03, 146.43, 141.28, 140.61, 140.13, 138.94, 137.64, 137.63, 137.16, 137.00, 136.48, 136.37, 135.06, 134.35, 132.94, 129.21, 128.73, 128.05, 127.96, 127.76, 126.96, 126.94, 126.79, 126.62, 126.48, 126.33, 126.09, 125.14, 124.54, 123.89, 123.54, 123.48, 122.46, 120.66, 120.34, 120.04, 119.44, 118.82, 118.31, 113.92, 110.01, 109.90, 72.33, 71.61, 46.87, 27.11.

### 6. HTL Polymer Preparation

In a glovebox, Monomer B2 (99.6% purity) (1.20 g, 1.45 mmol, 1.00 equiv) was dissolved in 7.00 mL anisole (electronic grade). Monomer A2 (25 mol %, 42 mol %) was added, followed by AIBN solution (0.20M in toluene, 1.09 mL, 0.218 mmol, 0.15 equiv). The mixture was heated to 70° C., and allowed to stir for 72 hours. The polymer was precipitated with methanol (60 mL) and isolated by filtration. The filtered solid was rinsed with additional portions of methanol (2×10 mL). The filtered solid was re-dissolved in anisole and the precipitation/filtration sequence was repeated twice more. The isolated solid was placed in a vacuum oven overnight at 50° C. to remove residual solvent. 1.23 g of white solid was isolated. Table 1 showed the polymer molecular weights and distributions for two polymers.

TABLE 1
Molecular weights of polymers with
different ratio of A and B monomers

| mole ratio (B:A) | Mₙ | M_{w} | M_{z} | M_{z+1} | M_{w}/Mₙ |
|---|---|---|---|---|---|
| 8:2 | 20,308 | 47,884 | 91,342 | 143,362 | 2.358 |
| 7:3 | 19,941 | 56,004 | 126,177 | 218,454 | 2.808 |

### 7. Light Emitting Device Fabrication

Indium tin oxide (ITO) glass substrates (2*2 cm) were cleaned with solvents ethanol, acetone, and isopropanol by sequence, and then were treated with a UV Ozone cleaner for 15 min. The hole injection layer (HIL) material Plexcore™ OC AQ-1200 from Plextronics Company was spin-coated from water solution onto the ITO substrates in glovebox and annealed at 150° C. for 20 min. After that, for comparative evaporative HTL, N-([1,1′-biphenyl]-4-yl)-9,9-dimethyl-N-(4-(9-phenyl-9H-carbazol-3-yl)phenyl)-9H-fluoren-2-amine, the substrate was transferred into a thermal evaporator for the deposition of the HTL, emitting materials layer (EML), electron transfer layer (ETL) and cathode; for inventive HTL for solution process, HTL materials (soluble copolymers) were deposited from anisole solution and annealed at 150° C. for 10 min to remove organic solvent. After that, the crosslinking of polymeric HTL was carried out on a hotplate in glovebox at 205° C. for 10 min. Then subsequent phosphorescent green (Ph-Green) EML, ETL and cathode were deposited in sequence. Finally these devices were hermetically sealed prior to testing.

To evaluate electroluminescent (EL) performances of the B-staged polymer as hole-transporting layer material, OLED devices with the following structures were fabricated:

Device A: ITO/AQ-1200/Comparative HTL (evaporated, 800 Å)/EML/ETL/Al;

Device B: ITO/AQ-1200/70:30 HTL copolymer (400 Å)/EML/ETL/Al;

Device C: ITO/AQ-1200/70:30 HTL copolymer with 10 wt. % trityl tetrakis(pentafluorophenyl)borate (400 Å)/EML/ETL/Al;

The thicknesses of HIL (AQ-1200), EML, ETL and cathode Al are 470, 400, 350 and 800 Å, respectively.

### III. Results

The current-voltage-luminance (J-V-L) characterizations for the OLED devices, that is, driving voltage (V), luminance efficiency (Cd/A), and international commission on illumination (CIE) data at 1000 nit and 50 mA/cm² luminance, and lifetime at 15000 nit for 10 hr were performed with a Keithly™ 238 High Current Source-Measurement Unit and a CS-100A Color and Luminance Meter from Konica Minolta Company and were listed in Table 2. Electroluminescence (EL) spectra of the OLED devices were collected by a calibrated CCD spectrograph and were fixed at 516 nm for all the four OLED device examples.

Device A was fabricated with evaporative Comparative HTL, while Device C was deposited with the inventive HTL copolymer comprising the additive (trityl tetrakis(pentafluorophenyl)borate from Acros Company) through a solution process. Device B was deposited with the inventive HTL copolymer without comprising the additive. Solution-processed Device C displayed comparable performance to the evaporative Device A.

TABLE 2

| | OLED J-V-L characterizations | | | | |
|---|---|---|---|---|---|
| Devices | V (1000 nit) | Cd/A | CIE | Lifetime | EL |
| A | 2.9 | 53.8 | 319, 628 | 98.4% | 516 nm |
| (evaporative) | | | | | |
| B (solution | 4.2 | 62.1 | 315, 629 | 71.1% | 516 nm |
| process) | | | | | |
| C (solution | 2.9 | 62.8 | 316, 628 | 95.8% | 516 nm |
| process) | | | | | |

What is claimed is:

1. A polymeric charge transfer layer composition comprising a polymer comprising, as polymerized units, at least one Monomer A and at least one Monomer B; wherein Monomer A has Structure A-I: 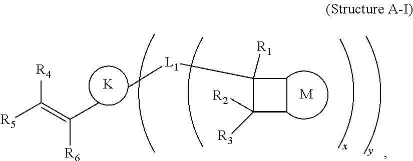
or Structure A-II: 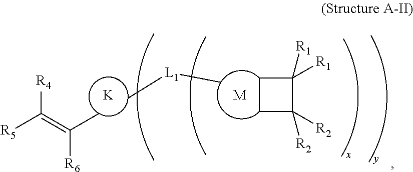
wherein K and M are each independently selected from substituted or unsubstituted aromatic moiety, substituted or unsubstituted heteroaromatic moiety, carbonyl moiety, and carboxy moiety; and
wherein L₁ is selected from heteroatom, aromatic moiety, heteroaromatic moiety, C₁-C₁₀₀ hydrocarbyl, C₁-C₁₀₀ substituted hydrocarbyl, C₁-C₁₀₀ heterohydrocarbyl, and C₁-C₁₀₀ substituted heterohydrocarbyl; and
wherein R₁ through R₆ are each independently selected from hydrogen, deuterium, hydrocarbyl, substituted hydrocarbyl, heterohydrocarbyl, substituted heterohydrocarbyl, halogen, cyano, aryl, substituted aryl, heteroaryl, substituted heteroaryl; and
wherein x and y are each independently an integer from 1 to 10; and
wherein Monomer B has Structure B: 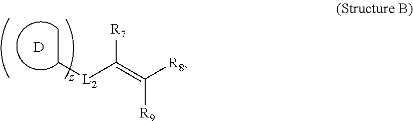
wherein D is selected from aromatic moiety, heteroaromatic moiety, C₁-C₁₀₀ hydrocarbyl, C₁-C₁₀₀ substituted hydrocarbyl, C₁-C₁₀₀ heterohydrocarbyl, and C₁-C₁₀₀ substituted heterohydrocarbyl; and
wherein L₂ may be absent, or is selected from aromatic moiety, heteroaromatic moiety, carboxy moiety, and carbonyl moiety; and
wherein R₇ through R₉ are each independently selected from hydrogen, deuterium, hydrocarbyl, substituted hydrocarbyl, heterohydrocarbyl, substituted heterohydrocarbyl, halogen, cyano, aryl, substituted aryl, heteroaryl, substituted heteroaryl; and
wherein z is an integer from 1 to 10.

2. The polymeric charge transfer layer according to claim 1 wherein Monomer A is selected from the following A1) through A14):

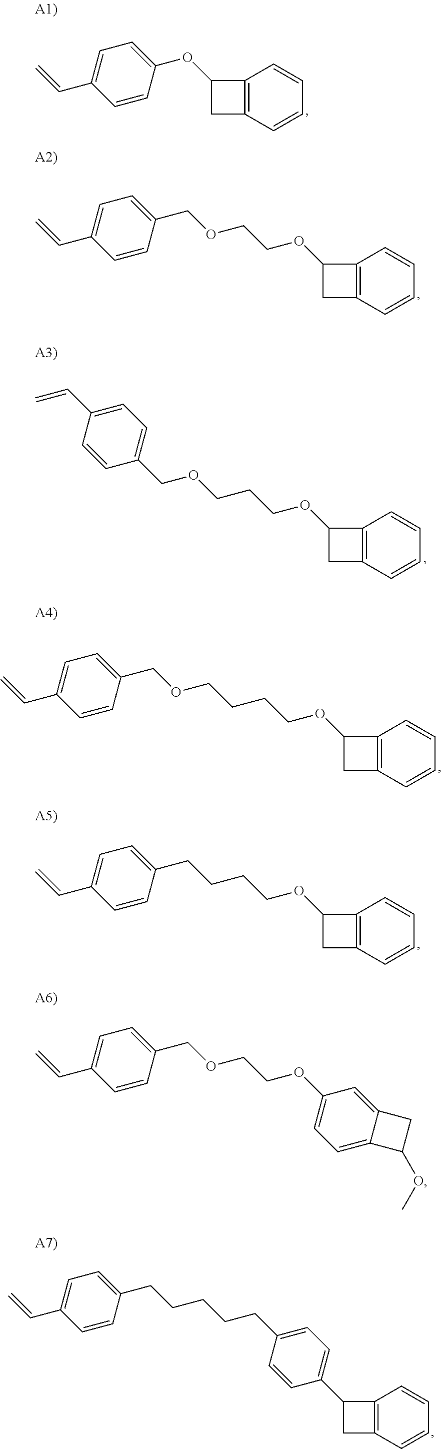

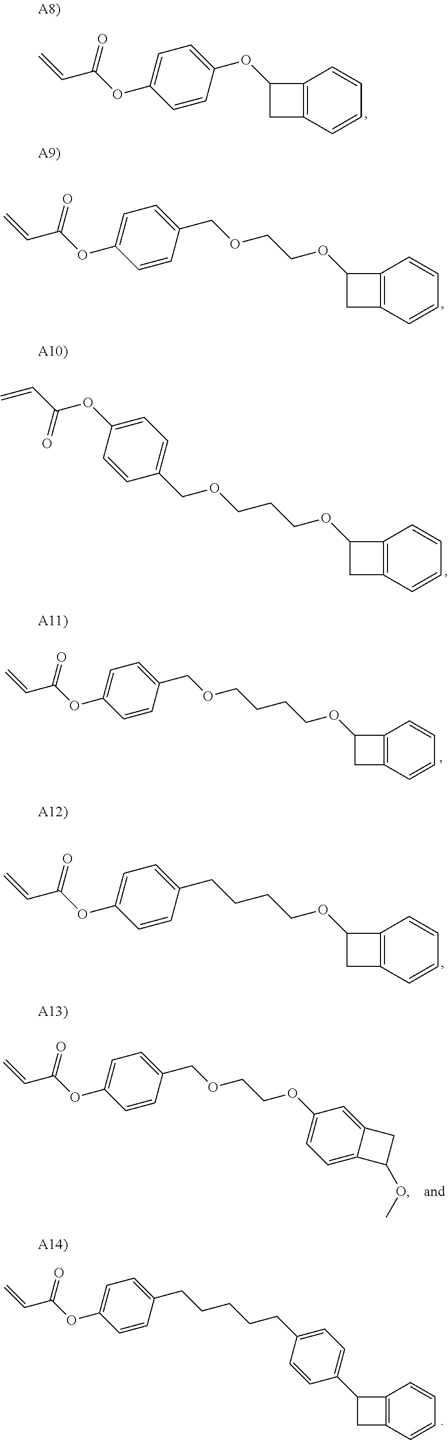

3. The polymeric charge transfer layer according to claim 1, wherein Monomer B is selected from the following B1) through B18):

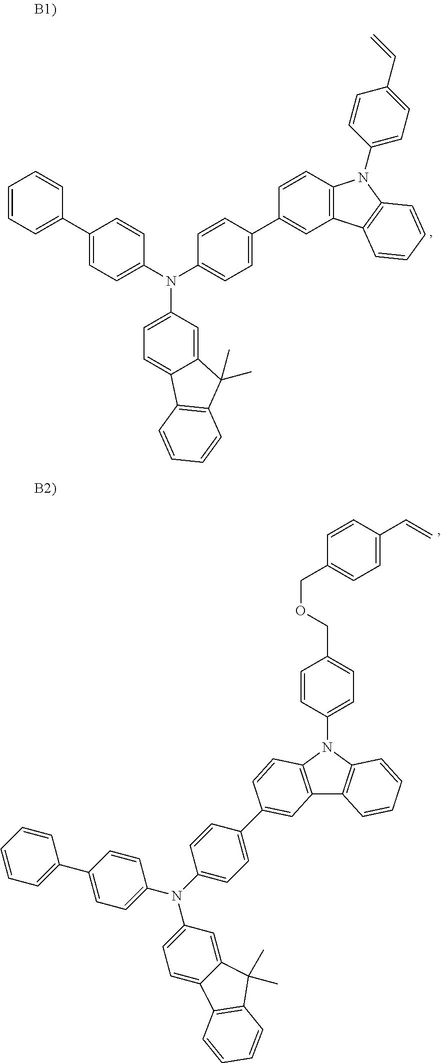 [SMILES: C=Cc1ccc(-n2c3ccccc3c3cc(-c4ccc(N(c5ccc(-c6ccccc6)cc5)c5ccc6c(c5)C(C)(C)c5ccccc5-6)cc4)ccc32)cc1 ; C=Cc1ccc(COCc2ccc(-n3c4ccccc4c4cc(-c5ccc(N(c6ccc(-c7ccccc7)cc6)c6ccc7c(c6)C(C)(C)c6ccccc6-7)cc5)ccc43)cc2)cc1]

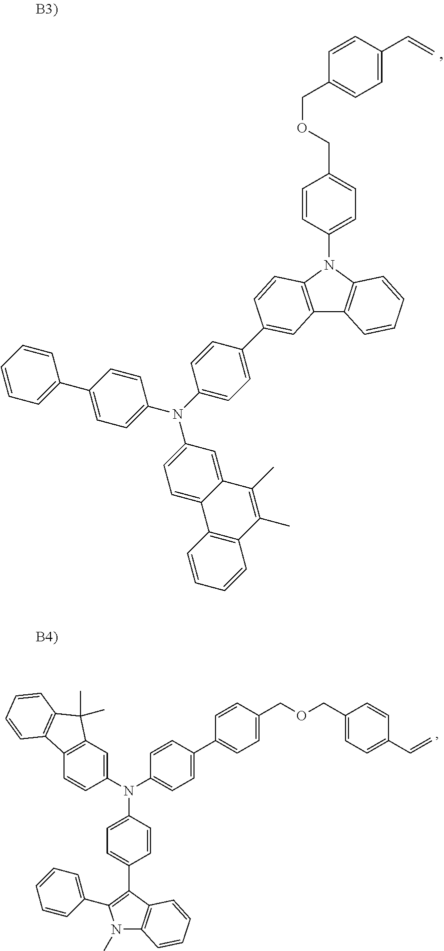 [SMILES: C=Cc1ccc(COCc2ccc(-n3c4ccccc4c4cc(-c5ccc(N(c6ccc(-c7ccccc7)cc6)c6ccc7c(c6)c(C)c(C)c6ccccc67)cc5)ccc43)cc2)cc1 ; C=Cc1ccc(COCc2ccc(-c3ccc(N(c4ccc(-c5c(-c6ccccc6)n(C)c6ccccc56)cc4)c4ccc5c(c4)C(C)(C)c4ccccc4-5)cc3)cc2)cc1]

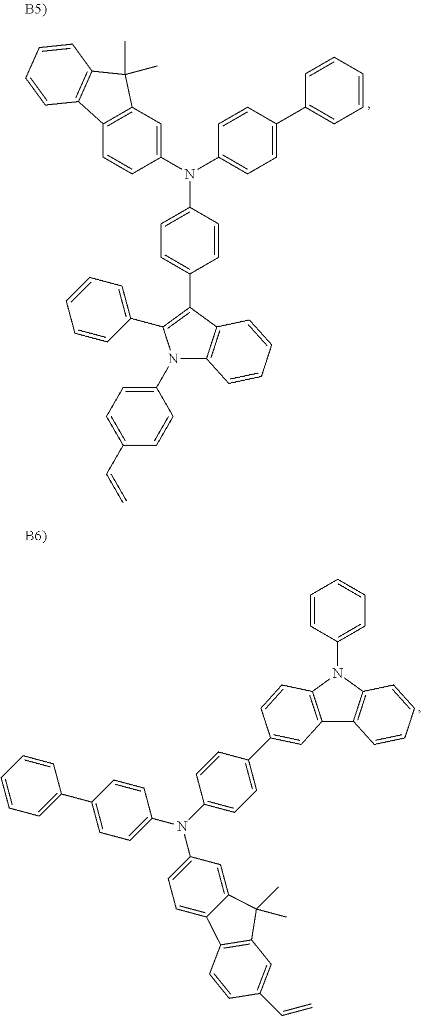 [SMILES: C=Cc1ccc(-n2c(-c3ccccc3)c(-c3ccc(N(c4ccc(-c5ccccc5)cc4)c4ccc5c(c4)C(C)(C)c4ccccc4-5)cc3)c3ccccc32)cc1 ; C=Cc1ccc2c(c1)C(C)(C)c1cc(N(c3ccc(-c4ccccc4)cc3)c3ccc(-c4ccc5c(c4)c4ccccc4n5-c4ccccc4)cc3)ccc1-2]

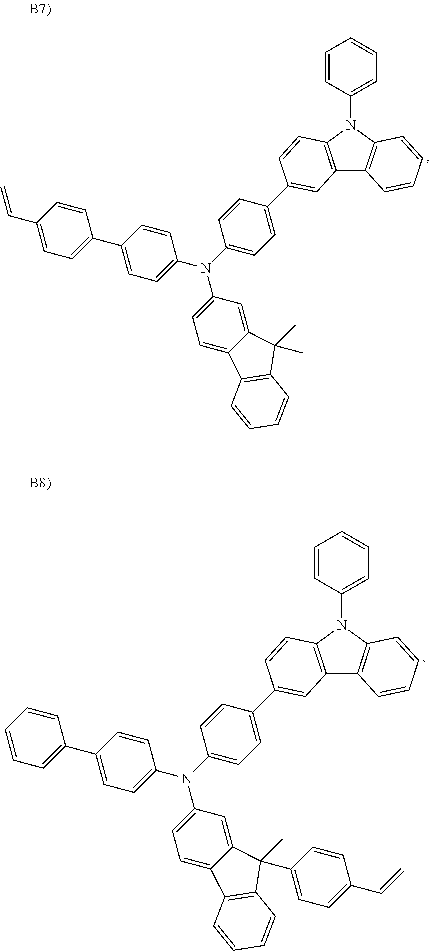 [SMILES: C=Cc1ccc(-c2ccc(N(c3ccc(-c4ccc5c(c4)c4ccccc4n5-c4ccccc4)cc3)c3ccc4c(c3)C(C)(C)c3ccccc3-4)cc2)cc1 ; C=Cc1ccc(C2(C)c3ccccc3-c3ccc(N(c4ccc(-c5ccccc5)cc4)c4ccc(-c5ccc6c(c5)c5ccccc5n6-c5ccccc5)cc4)cc32)cc1]

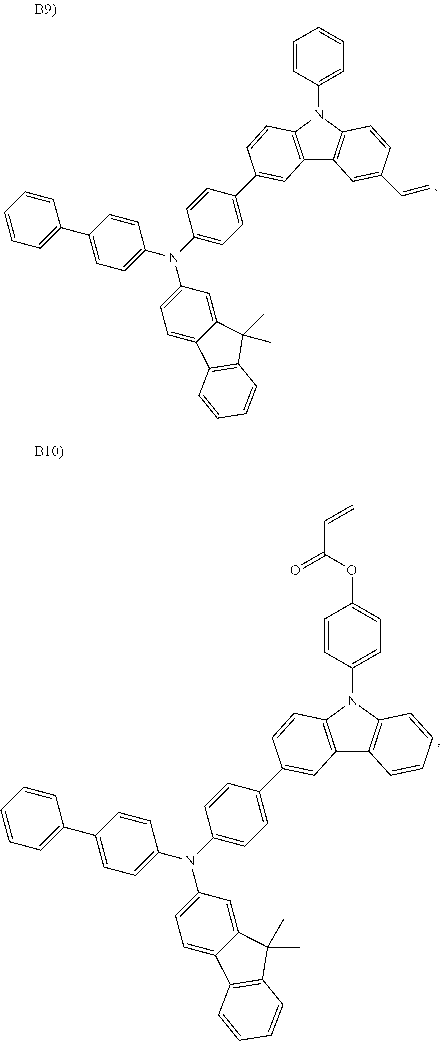 [SMILES: C=Cc1ccc2c(c1)c1cc(-c3ccc(N(c4ccc(-c5ccccc5)cc4)c4ccc5c(c4)C(C)(C)c4ccccc4-5)cc3)ccc1n2-c1ccccc1 ; C=CC(=O)Oc1ccc(-n2c3ccccc3c3cc(-c4ccc(N(c5ccc(-c6ccccc6)cc5)c5ccc6c(c5)C(C)(C)c5ccccc5-6)cc4)ccc32)cc1]

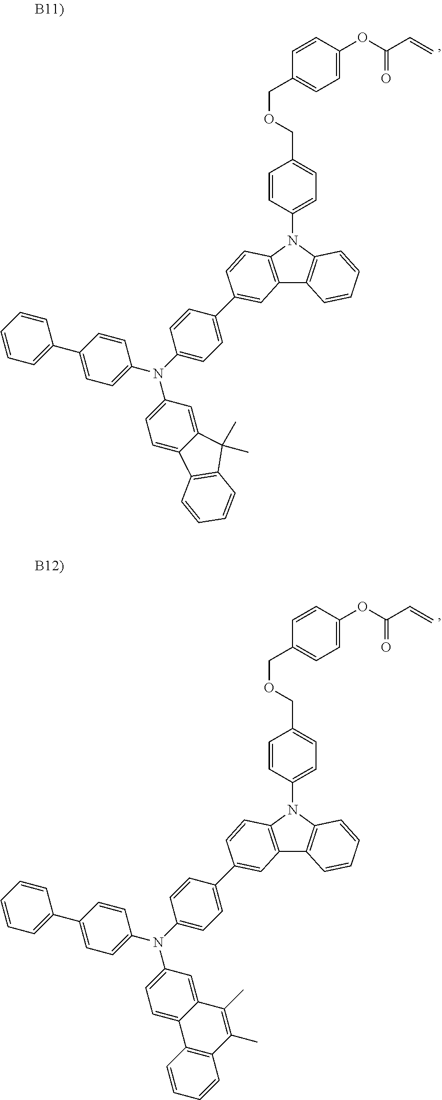 [SMILES: C=CC(=O)Oc1ccc(COCc2ccc(-n3c4ccccc4c4cc(-c5ccc(N(c6ccc(-c7ccccc7)cc6)c6ccc7c(c6)C(C)(C)c6ccccc6-7)cc5)ccc43)cc2)cc1 ; C=CC(=O)Oc1ccc(COCc2ccc(-n3c4ccccc4c4cc(-c5ccc(N(c6ccc(-c7ccccc7)cc6)c6ccc7c(c6)c(C)c(C)c6ccccc67)cc5)ccc43)cc2)cc1]

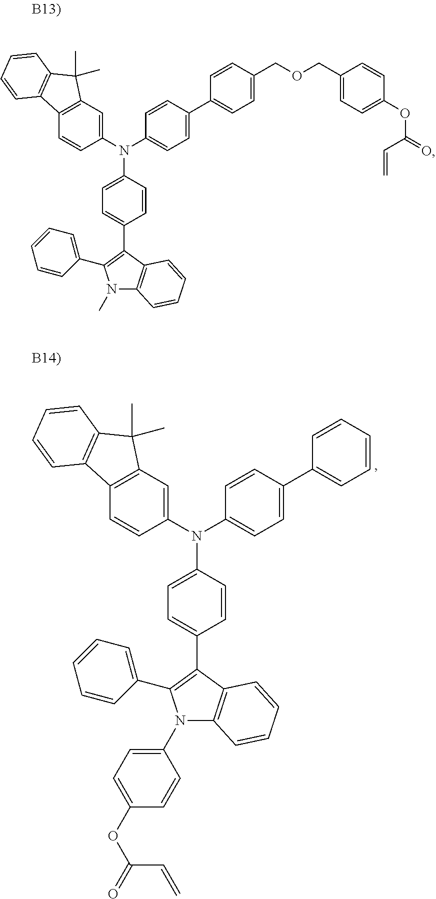 [SMILES: C=CC(=O)Oc1ccc(COCc2ccc(-c3ccc(N(c4ccc(-c5c(-c6ccccc6)n(C)c6ccccc56)cc4)c4ccc5c(c4)C(C)(C)c4ccccc4-5)cc3)cc2)cc1 ; C=CC(=O)Oc1ccc(-n2c(-c3ccccc3)c(-c3ccc(N(c4ccc(-c5ccccc5)cc4)c4ccc5c(c4)C(C)(C)c4ccccc4-5)cc3)c3ccccc32)cc1]

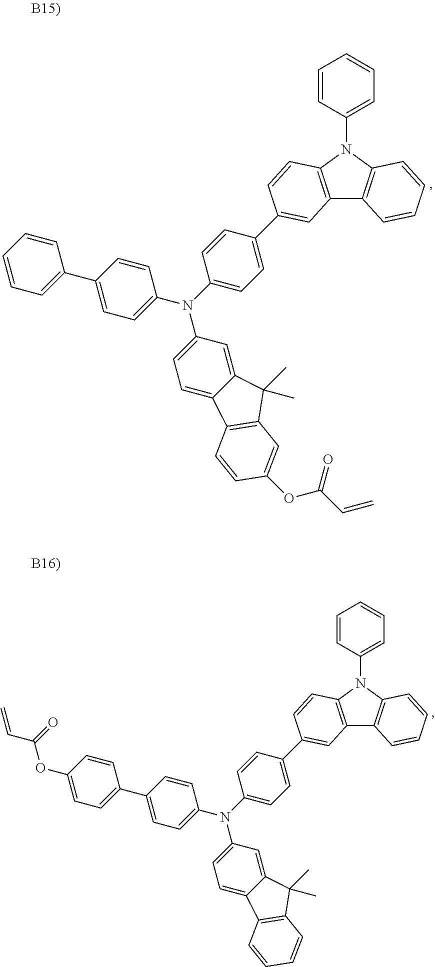 [SMILES: C=CC(=O)Oc1ccc2c(c1)C(C)(C)c1cc(N(c3ccc(-c4ccccc4)cc3)c3ccc(-c4ccc5c(c4)c4ccccc4n5-c4ccccc4)cc3)ccc1-2 ; C=CC(=O)Oc1ccc(-c2ccc(N(c3ccc(-c4ccc5c(c4)c4ccccc4n5-c4ccccc4)cc3)c3ccc4c(c3)C(C)(C)c3ccccc3-4)cc2)cc1]

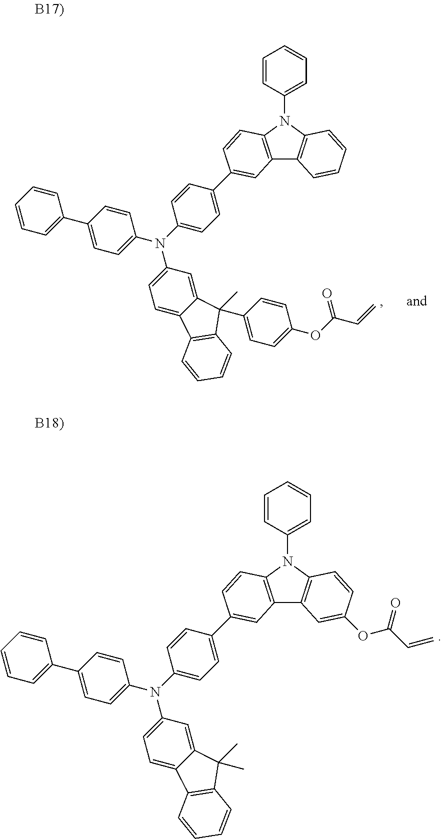 [SMILES: C=CC(=O)Oc1ccc(C2(C)c3ccccc3-c3ccc(N(c4ccc(-c5ccccc5)cc4)c4ccc(-c5ccc6c(c5)c5ccccc5n6-c5ccccc5)cc4)cc32)cc1 ; C=CC(=O)Oc1ccc2c(c1)c1cc(-c3ccc(N(c4ccc(-c5ccccc5)cc4)c4ccc5c(c4)C(C)(C)c4ccccc4-5)cc3)ccc1n2-c1ccccc1]

4. The polymeric charge transfer layer according to claim 1 wherein the molar ratio of all Monomer A to all Monomer B is from 0.1:99.9 to 99.9:0.1.

5. The polymeric charge transfer layer according to claim 1 further comprising an additive which is an organic salt component having Structure C:
[(R₁₀)_{q}-E-(R₁₁)ₜ]^{⊕}[G]^{⊖} (Structure C),
wherein E is an element selected from C, Si, Ge, Sn, Pb, N, P, As, Sb, Bi, O, S, Se, Te, and I; and
wherein G is a non-coordinating anion selected from borates, hexafluoroantimonate, hexafluoroarsenate, hexafluorophosphate, tetrakis(pentafluorophenyl)borate, and tetrafluoroborate; and
wherein R₁₀ and R₁₁ are each independently selected from hydrogen, deuterium, hydrocarbyl, substituted hydrocarbyl, heterohydrocarbyl, substituted heterohydrocarbyl, halogen, cyano, aryl, substituted aryl, heteroaryl, substituted heteroaryl, and
wherein q is an integer from 1 to v+1, and t is an integer from 0 to v−1; and
wherein v is the valence of the element E, and q+t=v+1.

6. The polymeric charge transfer layer according to claim 5 wherein the additive is from 0.01% to 50% by dry weight, based on total dry weight of the polymeric charge transfer layer composition.

7. The polymeric charge transfer layer according to claim 5 wherein the additive is selected from the following C1) through C10):

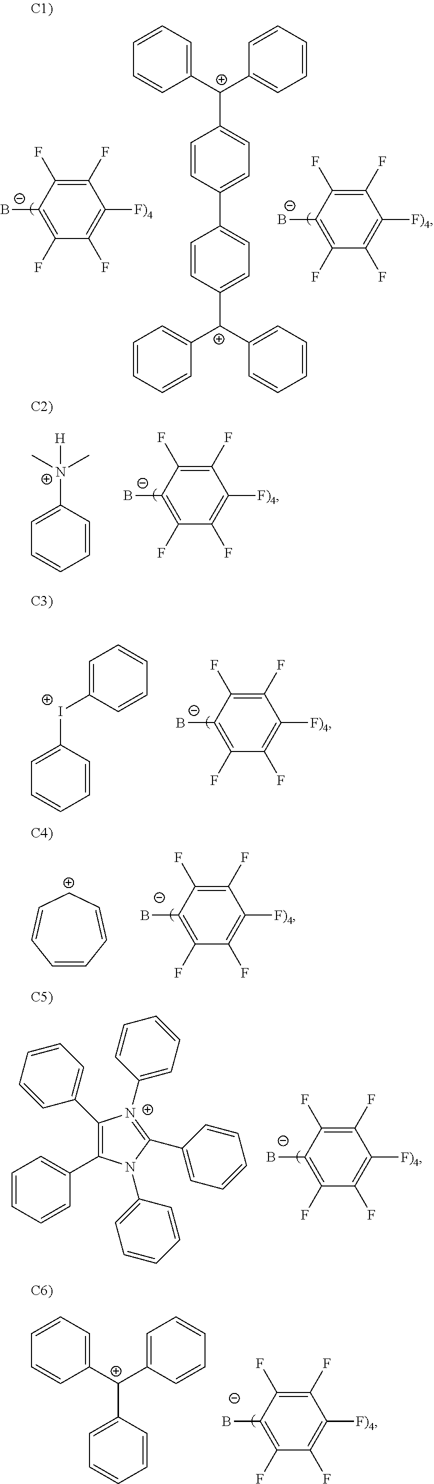

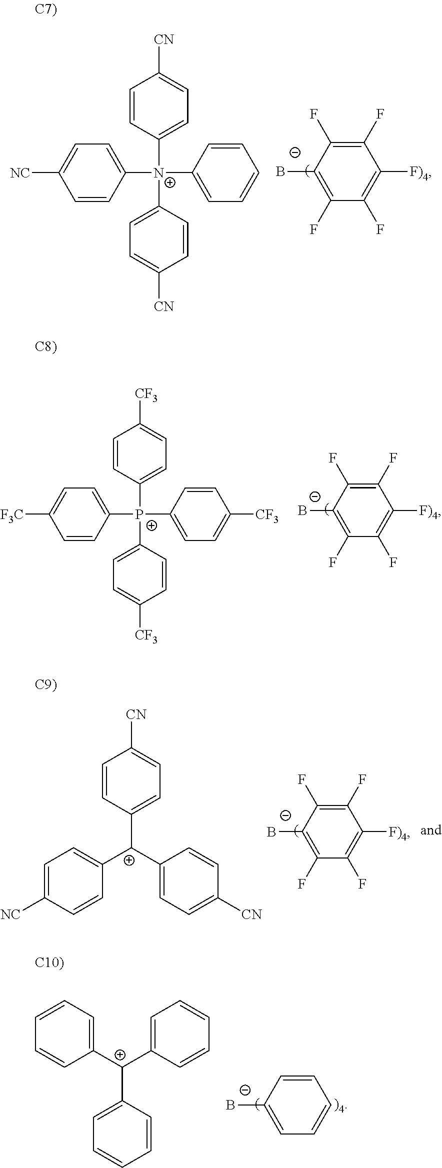

8. The polymeric charge transfer layer composition according to claim 1 wherein the polymeric charge transfer layer is a hole transfer layer.

9. An organic light emitting device comprising the polymeric charge transfer layer of claim 1.

10. An organic electronic device comprising the polymeric charge transfer layer of claim 1.
